(19) United States
(12) Patent Application Publication
(10) Pub. No.: US 2004/0224864 A1
(43) Pub. Date: Nov. 11, 2004

(54) Sterilized embolic compositions

(76) Inventors: William R. Patterson, Irvine, CA (US); Richard J. Greff, St. Pete Beach, FL (US); Richard S. Bein, San Clemente, CA (US)

Correspondence Address: Gerald F. Swiss, Foley & Lardner LLP, Three Palo Alto Square, 3000 El Camino Real, Suite 100, Palo Alto, CA, 94306-2121, US

(21) Appl. No.: 10/789,944

(22) Filed: Feb. 26, 2004

Related U.S. Application Data
(60) Provisional application No. 60/450,289, filed on Feb. 26, 2003.
(60) Provisional application No. 60/461,177, filed on Apr. 7, 2003.
(60) Provisional application No. 60/451,310, filed on Feb. 27, 2003.
(60) Provisional application No. 60/461,290, filed on Apr. 7, 2003.
(60) Provisional application No. 60/450,626, filed on Feb. 26, 2003.
(60) Provisional application No. 60/461,289, filed on Apr. 7, 2003.
(60) Provisional application No. 60/450,288, filed on Feb. 26, 2003.
(60) Provisional application No. 60/451,228, filed on Feb. 27, 2003.
(60) Provisional application No. 60/461,288, filed on Apr. 7, 2003.
(60) Provisional application No. 60/461,283, filed on Apr. 7, 2003.

(51) Int. Cl. A61K 7/50; C11D 1/00

(52) U.S. Cl. 510/161

(57) ABSTRACT

Disclosed are methods for sterilizing embolic compositions under conditions wherein the viscosity of the composition is minimally transformed after sterilization as compared to the composition prior to sterilization.

### CROSS-REFERENCE TO RELATED APPLICATIONS

This application claims the benefit under 35 USC §119(e) of United States Provisional Application Ser. No. 60/450,289, filed Feb. 26, 2003; No. 60/461,177, filed Apr. 7, 2003; No. 60/451,310, filed Feb. 27, 2003; 60/461,290, filed Apr. 7, 2003; No. 60/450,626, filed Feb. 26, 2003; No. 60/461,289, filed Apr. 7, 2003; No. 60/450,288, filed Feb. 26, 2003; No. 60/451,228, filed Feb. 27, 2003; No. 60/461,288, filed Apr. 7, 2003; No. 60/461,283, filed Apr. 7, 2003 which are hereby incorporated by reference in their entirety.

### BACKGROUND OF THE INVENTION

1. Field of the Invention

This invention is directed to methods for sterilizing embolic compositions under conditions wherein the sterilized composition exhibits a minimal change in its thixotropic behavior.

This invention is also directed to sterilized embolic compositions wherein the sterilized composition exhibits a minimal change in its thixotropic behavior as compared to composition prior to sterilization.

### REFERENCES

The following publications are cited and/or referenced in this application as superscript numbers:

¹ Greff, et al., U.S. Pat. No. 5,851,508, Compositions for Use in Embolizing Blood Vessels, issued Dec. 22, 1998.

² Whalen II, et al., U.S. Pat. No. 6,645,167, Methods for Embolizing Vascular Sites with an Embolic Composition, issued Nov. 11, 2003.

³ Whalen, II, et al., U.S. Pat. No. 6,531,111, High Viscosity Embolizing Compositions, issued Mar. 11, 2003.

⁴ S. C. Porter, U.S. Patent Application Publication No. 20030039696, Embolic Compositions with Non-cyanoacrylate Rheology Modifying Agents, published Feb. 27, 2003

⁵ C. Porter, et al., U.S. patent application Ser. No. 10/686,929, Polymeric Materials for Site Specific Delivery to the Body, filed Oct. 15, 2003

⁶ C. Porter, et al., U.S. patent application Ser. No. 10/687,545, Prepolymeric Materials for Site Specific Delivery to the Body, filed Oct. 15, 2003

⁷ Evans, et al., U.S. Pat. No. 5,695,480, Embolizing Compositions, issued Dec. 9, 1997.

⁸ Greff, U.S. patent application Ser. No. 10/162,653, Novel High Viscosity Embolic Compositions Comprising Prepolymers, filed Jun. 6, 2002.

⁹ Greff, et al., U.S. Pat. No. 6,248,800, Methods for Sterilizing Cyanoacrylate Compositions, issued Jun. 19, 2001.

¹⁰ Hademmenos & Massoud, (1998), The Physics of Cerebrovascular Diseases, Springer-Verlag, New York, USA.

¹¹ Bird, Stewart, & Lightfoot (1960), Transport Phenomena, John Wiley & Sons, New York, USA

¹² Braun & Rosen (2000), Rheology Modifiers Handbook: Practical Use and Application. William Andrew Publishing, New York, USA

¹³ Cabot Corp (2000), Cab-O-SIL Untreated Fumed Silica: Properties and Functions, Cabot Corp, Illinois, USA

¹⁴ Porter, U.S. Patent Application Publication No. 20020165582, Method and Apparatus for Delivering Materials to the Body, published Nov. 7, 2002

¹⁵ Casson (1959)Rheology of Dispersed Systems, Pergamon, N.Y.

¹⁶ Braun & Rosen (2000) Rheology Modifiers Handbook: practical use and application, William Andrew Publishing, NY.

¹⁷ Whalen, II, et al., Methods for Embolizing Vascular Sites with an Embolizing Composition, issued Nov. 11, 2003

All of the above publications are herein incorporated by reference in their entirety to the same extent as if each individual publication was specifically and individually indicated to be incorporated by reference in its entirety.

2. State of the Art

Embolization of blood vessels is conducted for a variety of purposes including the treatment of tumors, the treatment of lesions such as aneurysms, uncontrolled bleeding and the like.

Embolization of blood vessels is preferably accomplished via catheter techniques which permit the selective placement of the catheter at the vascular site to be embolized. In this regard, recent advancements in catheter technology as well as in angiography now permit neuroendovascular intervention including the treatment of otherwise inoperable lesions. Specifically, development of microcatheters and guide wires capable of providing access to vessels as small as 1 mm in diameter allows for the endovascular treatment of many lesions.

In one embodiment, embolizing compositions (embolic compositions) previously disclosed in the art include those comprising a biocompatible polymer, a biocompatible solvent and a contrast agent which allowed visualization of the in vivo delivery of the composition via fluoroscopy.¹⁷ In this embodiment, the use of high viscosity embolic compositions facilitates the formation of a contiguous or ball shape precipitate formed at the ejection port of a catheter situated in, for example, an aneurysm, thereby inhibiting outflow of the composition into the parent artery.³ Accordingly, highly viscous compositions provide better control of aneurysm embolization perceived angiographically as the ability to completely fill the neck of the aneurysm without leakage of the composition into the parent artery. The viscosity of these highly viscous compositions is preferably at least 500 cSt at 37° C. and wherein the viscosity of the composition is adjusted by the concentration and/or molecular weight of the biocompatible polymer employed.

Because of their high viscosity, specialty syringes and catheters are required to effect catheter delivery of these compositions due to the high shear stress required to effect delivery through the catheter.³ Accordingly, use of viscous embolic compositions exhibiting a Newtonian viscosity profile has a practical viscosity upper limit related more to the delivery equipment employed.

In order to enhance their deliverability, these embolic compositions have been formulated with one or more rheological modifiers which impart non-Newtonian viscosity profiles to these compositions. That is to say that under static conditions, an extremely high viscosity can be achieved. Contrarily, under shear stress conditions, the viscosity of these compositions is significantly lower thereby permitting delivery under acceptable injection pressures.⁵ In effect, the presence of the rheological modifier(s) permits the composition to exhibit a significantly higher viscosity under static conditions at a given temperature as compared to shear conditions at the same temperature. It is this differential viscosity characteristic that renders the non-Newtonian viscosity profiles to these compositions.

In another embodiment, the use of embolic compositions comprising a prepolymer and a contrast agent has been disclosed.⁷ Prepolymeric embolic compositions are often of low viscosity and, accordingly, exhibit undesirable flow properties in vivo. Specifically, low viscosity compositions can readily flow from the intended site such as an aneurysm or an arteriovenous malformation (AVM) into the parent artery where, upon polymerization, can cause unintended embolization of the parent artery. Methods for rendering these prepolymeric compositions less resistant to flow include the use of thickeners and/or rheological modifiers.^{4,6,8} In these cases, the thickener and/or rheological modifiers act to enhance the static viscosity such that the flow properties of the composition under static conditions is significantly reduced. Again, the presence of the rheological modifier imparts a non-Newtonian viscosity profile to the composition.

Particularly preferred rheological modifiers include hydroxyl-containing rheological modifiers and, in particular, amorphous, hydrophilic fumed silica which contains a plurality of silanol (Si—OH) surface groups.

When delivered in vivo by catheter techniques, the delivery protocol for the embolic composition often entails the use of an inflatable balloon which is used to inhibit blood flow during delivery. Since delivery can extend for prolonged periods of time, the inflatable balloon preferably transitions from a fully inflated to partially inflated position in order to inhibit ischemia at vascular sites distal to the site being treated.¹⁷ When the balloon is inflated, embolic composition is ejected from the distal portion of the catheter; whereas, when deflated or partially deflated, little or no embolic composition is ejected. Of course, ejection requires application of shear stress on the embolic composition and inhibition of ejection requires a decrease and preferably no shear stress on the composition. When the composition is pseudo-plastic, such repeated increases and decreases in shear stress do not materially change the delivery profile of the composition. However, if the composition exhibits thixotropic behavior during repeated increases and decreases in shear stress, the delivery profile does not remain constant and this poses a significant disadvantage to the reproducible delivery under similar delivery pressures.

In view of the above, it is important that the pseudo-plastic characteristics established for the composition prior to sterilization not materially change after sterilization and that the viscosity of the sterilized composition remain stable over prolonged times.

Both characteristics are essential in order to have predictable compositions for in vivo catheter delivery.

As to embolic compositions comprising a rheological modifier, attempts at heat sterilization of these embolic compositions have resulted in sterilized compositions having undesirable thixotropic properties and having an adverse effect on viscosity. For example, FIG. 1 illustrates the increase in viscosity arising in the heat sterilized embolic composition comprising a polymer solution together with a contrast agent and fumed silica as compared to the viscosity of the same composition prior to sterilization. Notwithstanding the non-Newtonian viscosity profile of the sterilized composition, this composition exhibits an increase in viscosity at high and low shear rates.

In assessing the basis for this change in viscosity behavior, the shear stress of the composition before and after sterilization was evaluated at different shear rates. The results of this evaluation show that the non-sterile embolic composition has little or no deviation in shear stress as the shear rate is varied from 0 to 250 s⁻¹ and from 250 to 0 s⁻¹. These results evidence pseudo-plastic behavior in the non-sterile composition.

However, for the heat sterilized sample, there is a much higher shear stress over 0 to 250 s⁻¹ then from 250 to 0 s⁻¹. This deviation, or hysteresis in shear stress as shear rate is increased then decreased, is thixotropy and is undesirable from a delivery point of view.

Without being limited to any theory, it is postulated that during heat sterilization, dehydration of the hydroxyl groups of the surface silanol moieties from different silica particles and/or hydroxyl groups of the biocompatible polymer results in covalent linkages rather than the reversible hydrogen bond linkages that existed in the non-sterile material. It is further postulated that this new structure is more viscous and requires more shear stress to flow. The dehydration is not predictable.

Moreover, extended shelf-life experiments demonstrate that heat sterilized compositions comprising hydroxyl-containing rheological modifiers are unstable over time with significant viscosity changes occurring.

In view of the above, it would be particular beneficial to provide for a sterilized embolic composition possessing properties similar to those found in the composition prior to sterilization.

### SUMMARY OF THE INVENTION

In one aspect, this invention is directed to methods for sterilizing embolic compositions. Specifically, this aspect of the invention is directed to the novel and unexpected result that sterilization of embolic compositions comprising hydroxyl-containing rheological modifier(s) using irradiation techniques provides sterilized compositions exhibiting minimal changes in its thioxotropic behavior as compared to the composition prior to sterilization with reduced variability from sterilized product to sterilized product. Moreover, the sterilized compositions of this invention are contemplated to exhibit a prolonged shelf life with limited changes in viscosity.

As noted above, the sterilized embolic compositions described herein comprise a hydroxyl-containing rheological modifier. When incorporated in sufficient amounts to impart shear thinning and pseudo-plastic properties to the composition, the presence of this rheological modifier materially changes the viscosity characteristics of the pre-sterilized composition at a given shear stress compared to the viscosity at static conditions. A reproducible curve illustrating the non-Newtonian relationship between the viscosity of the composition and the applied shear rate is established and is independent of the number of times the composition has been subject to shear increases or decreases.

Nevertheless, the sterilized compositions of this invention comprising a hydroxyl-containing rheological modifier exhibit reduced thixotropic behavior as compared to similar embolic compositions sterilized by thermal means.

Accordingly, in one of its method aspects, this invention is directed to a method for sterilizing an embolic composition comprising a hydroxyl-containing rheological modifier in an effective amount to impart shear thinning, pseudo-plastic properties to the composition which method comprises exposing the composition to a sufficient amount of irradiation to effect sterilization under conditions such that the sterilized composition exhibits a minimal change in its thixotropic behavior as compared to the composition prior to sterilization wherein such minimal change is characterized by an area between the two curves measuring shear stress at increasing and decreasing shear rates measured at from 0 to 250 s⁻¹ of no more than about 25,000 Pa/sec. More preferably, this area between the two curves is from about 1,000 to about 20,000 Pa/sec and, still more preferably, from about 1,500 to about 15,000 Pa/sec.

In one of its composition aspects, this invention is directed to a sterilized embolic composition comprising a hydroxyl-containing rheological modifier in an effective amount to impart shear thinning, pseudo-plastic properties to the composition wherein the sterilized composition exhibits a minimal change in its thixotropic behavior as compared to the composition prior to sterilization wherein such minimal change is characterized by an area between the two curves measuring shear stress at increasing and decreasing shear rates measured at from 0 to 250 s⁻¹ of no more than about 25,000 Pa/sec. More preferably, this area between the two curves is from about area 1,000 to about 20,000 Pa/sec and, still more preferably, from about 2,500 to about 15,000 Pa/sec.

In one preferred embodiment, the sterilized composition is further characterized by exhibiting a change of less than about 25% of its viscosity at 37° C. over a shelf-life of 6 months or more at a high shear of 250 sec⁻¹ as compared to the viscosity under the same conditions immediately after sterilization. More preferably, the change is less than 20%; even more preferably, the change is less than 15%; and still more preferably the change is less than 10%.

In one embodiment, the sterilized embolic composition further comprises a water insoluble, biocompatible polymer, a biocompatible solvent which dissolves the biocompatible polymer in the amounts employed and optionally a visualizing effective amount of a contrast agent. Preferably, this composition, in the absence of a rheological modifier, has a viscosity of at least 150 cP at 37° C. Even more preferably, this composition, in the absence of a rheological modifier, has a viscosity of at least 500 cP at 37° C.

In some aspects of this embodiment of the invention, somewhat more viscous compositions are provided. The sterilized composition comprises: (1) a biocompatible polymer that is insoluble in blood or other body fluids; (2) a biocompatible solvent that is miscible in blood and other body fluids and serves to solubilize the biocompatible polymer; (3) an optional biocompatible, water-insoluble contrast agent that is suspended in the composition; and (4) a sufficient amount of fumed silica or other hydroxyl-containing rheological modifier in the composition and to impart a shear thinning index to the composition (as that parameter is determined at 1.0 s⁻¹ and 10 s⁻¹ as set forth herein) of at least about 4 and preferably of from about 4.5 to 6.5.

This invention can be applied advantageously to relatively viscous compositions used to embolize aneurysms as well as the lower viscosity composition more typically employed in the treatment of an AVM and the like. The intermediate sterilized composition can serve both needs.

Representative high viscosity embodiments achieved using these sterilized embolic compositions have a viscosity of at least about 25,000 and especially at least about 50,000 cP at 0.24 s⁻¹ and 37° C. and a viscosity no greater than about 5,000 cP when a shear rate of at 100 s⁻¹ at 37° C. is applied. The viscosity of the intermediate viscosity, sterilized composition is at least about 4000 cP at 37° C. under at 0.24 s⁻¹ and no greater than about 2000 cP at 37° C. when a shear rate of at least about 100 s⁻¹ is applied. Preferably, these sterilized compositions are used to embolize a vascular site for the purpose of treating one or more of the following conditions; an aneurysm, an arteriovenous fistulae, uncontrolled bleeding and the like.

In yet another embodiment of the invention, a method for embolizing a vascular site in a mammal is provided. The method comprises delivering via a catheter into the vascular site a sterilized composition comprising (1) a biocompatible polymer that is insoluble in blood or other body fluids, (2) a biocompatible solvent that is miscible in blood and other body fluids and serves to solubilize the biocompatible polymer, (3) an optional biocompatible, water-insoluble contrast agent that is suspended in the composition, and (4) a sufficient amount of a rheological modifier in the composition to impart a shear thinning index to the composition of at least about 4 and preferably of from about 4.5 to 6.5. Upon delivery of the composition into the vascular site, the sterilized composition decelerates and its viscosity increases while forming a precipitate which embolizes the vascular site. One sterilized composition has a viscosity of at least about 25,000 and especially at least about 50,000 cP at 0.24 s⁻¹ and 37° C. and a viscosity no greater than about 5,000 cP when a shear rate of at 100 s⁻¹ at 37° C. is applied. The viscosity of the intermediate viscosity, sterilized composition is at least about 4000 cP at 37° C. under at 0.24 s⁻¹ and no greater than about 2000 cP at 37° C. when a shear rate of at least about 100 s⁻¹ is applied. Preferably, the vascular site in need of embolization is due to one of the following conditions; an aneurysm, arteriovenous fistulae, uncontrolled bleeding and the like.

A preferred sterilized composition which meets the viscosity requirement set forth in the composition and method just described comprises fumed silica and has a range of biocompatible polymer (weight/volume solvent) to fumed silica (weight/final weight) of from about 2.6 to 1 to about 3.6 to 1. More preferably, the ratio is from about 3.0 to 1 to about 3.2 to 1. Even more preferably, the ratio is about 3.1 to 1.

In another embodiment, another sterilized composition having a lower viscosity is provided. The sterilized composition comprises: (1) a biocompatible polymer that is insoluble in blood or other body fluids; (2) a biocompatible solvent that is miscible in blood and other body fluids and serves to solubilize the biocompatible polymer; (3) an optional biocompatible, contrast agent that is suspended in the composition; and (4) a sufficient amount of fumed silica or other hydroxyl-containing rheological modifier in the composition to impart a shear thinning index to the composition of at least about 4 and preferably of from about 4.5 to 6.5. The viscosity of the sterilized composition is at least about 2000 cP at 37° C. under at 0.24 s⁻¹ and no greater than about 500 cP at 37° C. when a shear rate of at least about 100 s⁻¹ is applied. Preferably, these compositions are used to embolize a vascular site for the purpose of treating one or more of the following conditions; arteriovenous malformations, tumors, uncontrolled bleeding and the like.

In still another embodiment of the invention, a method for embolizing a vascular site in a mammal is provided. The method comprises delivering via a catheter into the vascular site a sterilized composition comprising (1) a biocompatible polymer that is insoluble in blood or other body fluids, (2) a biocompatible solvent that is miscible in blood and other body fluids and serves to solubilize the biocompatible polymer, (3) an optional biocompatible, contrast agent that is suspended in the composition, and (4) a sufficient amount of fumed silica or other hydroxyl-containing rheological modifier in the composition. Upon delivery of the composition into the vascular site, the sterilized composition decelerates and its viscosity increases while forming a precipitate which embolizes the vascular site. In this embodiment, however, viscosity of the composition is at least about 2000 cP at 37° C. under at 0.24 s⁻¹ and no greater than about 500 cP at 37° C. when a shear rate of at least about 100 s⁻¹ is applied. Preferably, the vascular site in need of embolization is due to one of the following conditions; arteriovenous malformation, tumor, uncontrolled bleeding and the like.

In another embodiment, the sterilized embolic composition comprises a prepolymer, an effective amount of a hydroxyl-containing rheological modifier to impart a viscosity to the composition of at least 1,000 cP at 37° C. and 0.24 sec⁻¹ and optionally a visualizing effective amount of a contrast agent wherein the prepolymer, upon polymerization, forms a water insoluble, biocompatible polymer. Preferably, this composition has a viscosity of no more 150 cP at 37° C. in the absence of a rheological modifier. Even more preferably, this composition has a viscosity of no more than 100 cP at 37° C. in the absence of a rheological modifier.

In still another embodiment of the invention, a method for embolizing a vascular site in a mammal is provided. The method comprises delivering via a catheter into the vascular site a sterilized composition comprising (1) a biocompatible prepolymer which forms a polymer in vivo that is insoluble in blood or other body fluids, (2) an optional biocompatible solvent that is miscible in blood and other body fluids, (3) an optional biocompatible contrast agent that is suspended in the composition, and (4) a sufficient amount of fumed silica or other hydroxyl-containing rheological in the composition. Upon delivery of the composition into the vascular site, the sterilized composition decelerates and its viscosity increases while forming a precipitate which embolizes the vascular site. In this embodiment, however, viscosity of the composition is at least about 1000 cP at 37° C. and 0.24 s⁻¹ and no greater than about 150 cP at 37° C. when a shear rate of at least about 100 s⁻¹ is applied. Preferably, the vascular site in need of embolization is due to one of the following conditions; arteriovenous malformation, tumor, uncontrolled bleeding and the like.

In one embodiment, the sterilizing irradiation is gamma irradiation. In another embodiment, the sterilizing irradiation is electron beam (e-beam) irradiation. In either case, a sufficient dosage of the irradiation is employed to effect sterilization of the composition.

Preferably, the hydroxyl-containing rheological modifier is amorphous, hydrophilic, fumed silica.

In another embodiment, this invention is directed to a method for sterilizing an embolic composition comprising a hydroxyl-containing rheological modifier in an amount sufficient to impart shear thinning and pseudo-plastic properties to the composition under conditions wherein the sterilized composition exhibits a minimal increase in its thixotropic behavior as compared to the composition prior to sterilization which method comprises selecting an embolic composition comprising a hydroxyl-containing rheological modifier wherein at least about 25% of the surface hydroxyl groups have been converted to non-hydroxyl groups and sterilizing said composition such that the sterilized composition exhibits a minimal change its thixotropic behavior as compared to the composition prior to sterilization which such minimal change is characterized by an area between the two curves measuring shear stress at increasing and decreasing shear rates measured at from 0 to 250 s⁻¹ of no more than about 25,000 Pa/sec. More preferably, this area between the two curves is from about area 1,000 to about 20,000 Pa/sec and, still more preferably, from about 2,500 to about 15,000 Pa/sec.

Sterilization, in this case, can be effected by heat or irradiation such as gamma irradiation or e-beam irradiation.

Preferably, the hydroxyl-containing rheological modifier is amorphous, hydrophilic fumed silica.

In one of its composition aspects, this invention is directed to a sterilized embolic composition comprising a sufficient amount of a hydroxyl-containing rheological modifier to impart pseudo-plastic, shear thinning properties to the composition wherein at least about 25% of the surface hydroxyl groups have been converted to non-hydroxyl groups and further wherein said sterilized composition exhibits a minimal change in its thixotropic behavior as compared to the composition prior to sterilization wherein such minimal change is characterized by an area between the two curves measuring shear stress at increasing and decreasing shear rates measured at from 0 to 250 s⁻¹ of no more than about 25,000 Pa/sec. More preferably, this area between the two curves is from about area 1,000 to about 20,000 Pa/sec and, still more preferably, from about 2,500 to about 15,000 Pa/sec.

In a preferred embodiment, at least about 50% of the surface hydroxy groups have been converted to non-hydroxyl groups and, even more preferably, at least about 90% of the surface hydroxyl groups have been converted to non-hydroxyl groups. Still more preferably, at least 98% of the surface hydroxyl groups have been converted to non-hydroxyl groups. When amorphous, hydrophilic fumed silica is employed, the surface hydroxyl groups are silanol (Si—OH) groups which are preferably converted to non-silanol groups (e.g., siloxane groups) in the amounts as described above.

In one preferred embodiment, the sterilized composition is further characterized by exhibiting a change of less than about 25% of its viscosity at 37° C. over a shelf-life of 6 months or more at a high shear of 250 sec⁻¹ as compared to the viscosity under the same conditions immediately after sterilization. More preferably, the change is less than 20%; even more preferably, the change is less than 15%; and still more preferably the change is less than 10%.

In one embodiment, the sterilized embolic composition comprises a water insoluble, biocompatible polymer, a biocompatible solvent which dissolves the biocompatible polymer in the amounts employed and optionally a visualizing effective amount of a contrast agent. Preferably, this composition, in the absence of the rheological modifier, has a viscosity of at least 150 cP at 37° C. Even more preferably, this composition, in the absence of a rheological modifier, has a viscosity of at least 500 cP at 37° C.

In some aspects of this embodiment of the invention, somewhat more viscous compositions are provided. These sterilized compositions comprise: (1) a biocompatible polymer that is insoluble in blood or other body fluids; (2) a biocompatible solvent that is miscible in blood and other body fluids and serves to solubilize the biocompatible polymer; (3) an optional biocompatible, water-insoluble contrast agent that is suspended in the composition; and (4) a sufficient amount of fumed silica or other hydroxyl-containing rheological modifier having at least 25% of the surface hydroxyl groups converted to non-hydroxyl groups to impart a shear thinning index to the composition (as that parameter is determined at 1.0 s⁻¹ and 10 s⁻¹ as set forth herein) of at least about 4 and preferably of from about 4.5 to 6.5.

This invention can be applied advantageously to relatively viscous, sterilized compositions used to embolize aneurysms as well as the lower viscosity composition more typically employed in the treatment of an AVM and the like. The intermediate viscosity sterilized composition can serve both needs.

Representative high viscosity embodiments achieved using this invention have a viscosity of at least about 25,000 and especially at least about 50,000 cP at 0.24 s⁻¹ and 37° C. and a viscosity no greater than about 5,000 cP when a shear rate of at 100 s⁻¹ at 37° C. is applied. The viscosity of the intermediate viscosity sterilized composition is at least about 4000 cP at 37° C. and at a minimal shear of 0.24 s⁻¹ and no greater than about 2000 cP at 37° C. when a shear rate of at least about 100 s⁻¹ is applied. Preferably, these sterilized compositions are used to embolize a vascular site for the purpose of treating one or more of the following conditions; an aneurysm, an arteriovenous fistulae, uncontrolled bleeding and the like.

In yet another embodiment of the invention, a method for embolizing a vascular site in a mammal is provided. The method comprises delivering via a catheter into the vascular site a sterilized composition comprising (1) a biocompatible polymer which forms a polymer in vivo that is insoluble in blood or other body fluids, (2) a biocompatible solvent that is miscible in blood and other body fluids and serves to solubilize the biocompatible polymer, (3) an optional biocompatible, water-insoluble contrast agent that is suspended in the composition, and (4) a sufficient amount of fumed silica or other hydroxyl-containing rheological modifier having at least 25% of the surface hydroxyl groups converted to non-hydroxyl groups to impart a shear thinning index to the composition of at least about 4 and preferably of from about 4.5 to 6.5. Upon delivery of the composition into the vascular site, the composition decelerates and its viscosity increases while forming a precipitate which embolizes the vascular site. One composition has a viscosity of at least about 25,000 and especially at least about 50,000 cP at 0.24 s⁻¹ and 37° C. and a viscosity no greater than about 5,000 cP when a shear rate of at 100 s⁻¹ at 37° C. is applied. The viscosity of the intermediate viscosity composition is at least about 4000 cP at 37° C. and 0.24 s⁻¹ and no greater than about 2000 cP at 37° C. when a shear rate of at least about 100 s⁻¹ is applied. Preferably, the vascular site in need of embolization is due to one of the following conditions; an aneurysm, an arteriovenous fistula, uncontrolled bleeding and the like.

A preferred sterilized composition which meets the viscosity requirement set forth in the composition and method just described comprises fumed silica as the hydroxyl-containing rheological modifier and has a range of biocompatible polymer (weight/volume solvent) to fumed silica (weight/final weight) of from about 2.6 to 1 to about 3.6 to 1. More preferably, the ratio is from about 3.0 to 1 to about 3.2 to 1. Even more preferably, the ratio is about 3.1 to 1.

In another embodiment, another sterilized composition having a lower viscosity is provided. The sterilized composition comprises: (1) a biocompatible polymer that is insoluble in blood or other body fluids; (2) a biocompatible solvent that is miscible in blood and other body fluids and serves to solubilize the biocompatible polymer; (3) an optional biocompatible, water-insoluble contrast agent that is suspended in the composition; and (4) a sufficient amount of fumed silica or other hydroxyl-containing rheological modifier having at least 25% of the surface hydroxyl groups converted to non-hydroxyl groups to impart a shear thinning index to the composition of at least about 4 and preferably of from about 4.5 to 6.5. The viscosity of the composition is at least about 2000 cP at 37° C. and at 0.24 s⁻¹ and no greater than about 500 cP at 37° C. when a shear rate of at least about 100 s⁻¹ is applied. Preferably, these compositions are used to embolize a vascular site for the purpose of treating one or more of the following conditions; arteriovenous malformations, tumors, uncontrolled bleeding and the like.

In still another embodiment of the invention, a method for embolizing a vascular site in a mammal is provided. The method comprises delivering via a catheter into the vascular site a sterilized composition comprising (1) a biocompatible polymer that is insoluble in blood or other body fluids, (2) a biocompatible solvent that is miscible in blood and other body fluids and serves to solubilize the biocompatible polymer, (3) an optional biocompatible, water-insoluble contrast agent that is suspended in the composition, and (4) a sufficient amount of fumed silica or other hydroxyl-containing rheological modifier having at least 25% of the surface hydroxyl groups converted to non-hydroxyl groups. Upon delivery of the composition into the vascular site, the composition decelerates and its viscosity increases while forming a precipitate which embolizes the vascular site. In this embodiment, however, viscosity of the composition is at least about 2000 cP at 37° C. under at 0.24 s⁻and no greater than about 500 cP at 37° C. when a shear rate of at least about 100 s⁻¹ is applied. Preferably, the vascular site in need of embolization is due to one of the following conditions; arteriovenous malformation, tumor, uncontrolled bleeding and the like.

In another embodiment, the sterilized embolic composition comprises a prepolymer and an optional visualizing effective amount of a contrast agent wherein the prepolymer, upon polymerization, forms a water insoluble, biocompatible polymer. Preferably, this composition has a viscosity of no more than 150 cP at 37° C. in the absence of a rheological modifier. Even more preferably, this composition has a viscosity of no more than 100 cP at 37° C. in the absence of a rheological modifier. As above, this composition further comprises a hydroxyl-containing rheological modifier in an effective amount to impart a static viscosity to the composition of at least 1,000 cP at 37° C.

### BRIEF DESCRIPTION OF THE DRAWING

FIG. 1 illustrates the change in non-Newtonian viscosity versus shear rate relationship for an embolic composition comprising amorphous, hydrophilic, fumed silica as a rheological modifier both before and after sterilization.

FIG. 2 illustrates the presence of surface hydroxyl groups in the silanol portion of amorphous, hydrophilic, fumed silica.

FIG. 3A illustrates the hydrogen bonding between two surface hydroxyl groups of the silanol portion of amorphous, hydrophilic, fumed silica whereas FIG. 3B illustrates the network of silica particles arising under static conditions due to the hydrogen bonding illustrated in FIG. 3A.

FIG. 4 illustrates a mixing chamber for use in mixing an embolic composition comprising a hydroxyl-containing silica rheological modifier and the vortices formed during mixing.

FIG. 5 illustrates the non-Newtonian viscosity versus shear rate relationship of four different embolic compositions comprising amorphous, hydrophilic, fumed silica over a variety of shear rates.

FIG. 6 illustrates the main effects plot showing contributing curves of the input variables (weight/final weight) for control at the neck of the aneurysm. The horizontal line represents the highest average score generated for the neck control response variable.

FIG. 7A illustrates the properties of a composition identified as Formula K in a Casson plot.

FIG. 7B illustrates Formula K in a Power Law plot.

FIG. 8A illustrates a cross-section of a canine carotid artery which was embolized using an embolic composition not containing fumed silica.

FIG. 8B illustrates a cross-section of a canine carotid artery which was embolized using an embolic composition of this invention.

FIG. 9 illustrates in schematic partially exploded side view a device for delivering the rheologically-modified compositions. In this view the parts involved in filling a special vented-barrel syringe with composition provided in an aluminum squeeze tube are shown.

FIG. 10 illustrates in schematic partially exploded side view the device depicted in FIG. 10 now configured to accurately and controllably administer the rheologically-modified composition to a catheter for delivery to a point of use.

FIG. 11 illustrates in a side view a puncture cap and syringe interface which is part of the syringe filling set up depicted in FIG. 9.

FIG. 12 illustrates in a cross-sectional side view the interface depicted in FIG. 11.

FIG. 13 illustrates in a side view the vented barrel syringe body which makes up part of the systems shown in FIG. 9 and FIG. 10.

FIG. 14 illustrates in distal-end view the syringe body showing its generally oblong handle.

FIG. 15 through FIG. 25 all illustrate various aspects of a “Quick Stop” mechanism which permits the immediate coupling and decoupling of a lead screw drive mechanism for accurately, manually controlling the delivery of the rheologically-modified compositions from a syringe barrel.

More particularly FIG. 15 illustrates a generally top and side perspective view of the Quick Stop mechanism.

FIG. 16 illustrates a generally bottom and end perspective view of the Quick Stop mechanism.

FIG. 17 illustrates in side cross-sectional view the Quick Stop mechanism shown engaging the oblong handle or flange of the syringe barrel illustrated in FIG. 13 and FIG. 14.

FIG. 18 illustrates in bottom perspective view the Quick Stop mechanism and shows how the parts of the mechanism move relative to one another to engage and disengage the lead screw.

FIG. 19 illustrates a bottom view of the Quick Stop mechanism.

FIG. 20 illustrates a bottom view of the base of the Quick Stop mechanism.

FIG. 21 illustrates an end view of the base of the Quick Stop mechanism.

FIG. 22 illustrates a cross-sectional side view of the base of the Quick Stop mechanism.

FIG. 23 illustrates a bottom view of the activator of the Quick Stop mechanism.

FIG. 24 illustrates a side view of the activator.

FIG. 25 illustrates in top view, the threaded pincher which grips and releases the threaded syringe plunger to effect the Quick Stop function.

FIGS. 26A-E illustrate the rheological properties of sterilized rheologically-modified compositions against control.

### DETAILED DESCRIPTION OF THE PREFERRED EMBODIMENTS

This invention is directed, in part, to sterilized embolic compositions wherein the sterilized composition exhibits a minimal change in its thixotropic behavior as compared to the composition prior to sterilization. This invention is also directed to methods for preparing such sterile embolic compositions.

Before the present invention is described in further detail, it is to be understood that unless otherwise indicated this invention is not limited to any particular composition or hydroxyl-containing rheological modifier as such may vary. It is also to be understood that the terminology used herein is for the purpose of describing particular embodiments only and is not intended to limit the scope of the present invention.

It must be noted that as used herein and in the claims, the singular forms “a,” “and” and “the” include plural referents unless the context clearly dictates otherwise.

In this specification and in the claims which follow, reference will be made to a number of terms which shall be defined to have the following meanings:

The term “alkylene” refers to divalent alkyl groups of from 1 to 20 carbon atoms, which may be straight chained or branched, and include, for example, methylene (—CH₂—) ethylene (—CH₂CH₂—), n-propylene (—CH₂CH₂CH₂—), iso-propylene [—CH(CH₃)CH₂— and —CH₂CH(CH₃)—] and the like.

“Viscosity” (represented by the Greek letter eta, □) is an inherent property of a fluid that exerts a resistance against the movement of the fluid.¹⁰ Non-Newtonian fluids exhibit an “apparent viscosity”. The common unit of viscosity is centipoise (cP) and the International System of Units (SI) unit is the pascal second or Pa*s.

“Shear rate” (represented by the Greek letter γ, technically, a dot is placed over the letter but will not be done in this application), is proportional to the fluid flow rate. Conceptually, then, the higher the flow rate of a fluid through, e.g., a catheter, the higher the shear rate experienced by the fluid. The unit of shear rate is the reciprocal second, or 1/s or s⁻¹.

“Shear stress” (represented by the Greek letter tau, τ) of a fluid is the force per unit area (i.e. pressure) that must be applied to maintain the movement of a fluid. The unit of shear stress is the pascal, or Pa.

Newton's Law of Viscosity relates shear stress to shear rate in the following manner:

“Yield stress” is the force per unit area (i.e. pressure) that must be applied to move the fluid from rest. The unit of yield stress is the pascal, or Pa.

“Rheology” is the science of deformation and flow and includes the study of the mechanical properties of gases, liquids, plastics, asphalts, and crystalline materials.¹¹

“Shear Thinning Index” is the degree of shear thinning over a range of shear rates or rotational speeds. Higher ratios indicate greater shear thinning. Shear thinning index, as used herein, is calculated by dividing the apparent viscosity at 1 s⁻¹ by the apparent viscosity at 10 s⁻¹.

“Rheology modifiers” or “rheological modifiers” as used herein refers to a component which when added to the polymer, solvent, and contrast agent composition imparts high rest viscosity or yield stress of the composition (e.g., greater than about 1000 cP at 37° C. at 0.24 s⁻¹ shear rate or a yield stress greater than about 10 Pa measured from 37° C. shear stress versus shear rate data by the method of Casson) but permits the composition to readily flow under shear stress. Preferably, the rheological modifier is fumed silica.

The term “rheologically-modified compositions” refers to compositions comprising the biocompatible polymer, biocompatible solvent, contrast agent and a rheological modifier as described above.

“Hydroxyl-containing rheological modifiers” are rheological modifiers containing surface hydroxyl functionality in any form including, by way of example only, silanol (—Si—OH), carboxyl [—C(O)OH], alkylene-OH, phosphates [—OP(O)₂OH], and the like.

Hydroxyl-containing rheological modifiers include by way of example only, polymers such as poly(acrylates) such as poly(2-hydroxyethylacrylates), poly(alkenes) such as copolymers of ethylene and maleic acid, polyvinylalcohol, oxidized poly(alkenes), cellulosic polymers and copolymers [including hydroxypropylcellulose, hydroxypropylmethylcellulose, carboxymethylcellulose, sodium hydroxyethylcellulose, hydroxyethylcellulose and methylcellulose], poly(methacrylates) such as poly(2-hydroxyethylmethacrylates), poly(saccharides), poly(siloxanes), carrageenan, guar, xanthan gum, locus bean gum, homo- and co-polymers of mannuronic acid and glucuronic acid, and the like. To the extent that these polymers may be soluble in the biocompatible solvent of a polymeric embolic composition and/or in the prepolymer of the prepolymeric embolic composition, these polymers are sometimes referred to herein as “soluble rheological modifiers”.

One with skill in the art will be able to determine the amount of polymer to be included in the embolic composition based on the relative weight of the polymer and the desired viscosity of the liquid composition. Typically, the polymers will have molecular weight of above 75,000. More preferably, the polymer will have a molecular weight of greater than 200,000. The polymer can be included in a liquid medium, which can comprise either the polymeric embolic solution or the prepolymer liquid.

The rheology modifying agent also includes fine, inorganic hydroxyl-containing particulate materials which are sometimes referred to herein as “particulate rheological modifiers”. These rheology modifying agents alter the rheological and cohesive properties of the embolic composition. The inorganic particulate material may be selected from the group consisting of hydrophilic fumed silica, silicatious earths, for example, bentonite, or other inorganic particulate gelling or suspending materials capable of altering the rheology of the embolic composition such as organoclays, water-swellable clays, and the like. The size and concentration of these particulate rheology modifying agents can be selected from a broad range of such suitable particulate materials provided that the particulate materials impart non-Newtonian viscosity profiles to the embolic composition. Suitable materials can include, for example, amorphous, hydrophilic fumed silica particles of about 10 nanometers (0.1 micron) in diameter, and generally can comprise particles of less than about 5 microns in diameter, depending on the nature of the particle selected.

Embolizing compositions comprising an inorganic particulate rheology modifying agent will exhibit a change in apparent viscosity upon moving from an environment with a first hydrodynamic shear rate to an environment having a second hydrodynamic shear rate. The effect sought is typically referred to as “shear thinning behavior.” For example, the embolizing composition has a low apparent viscosity when flowing through a microcatheter and a relatively high apparent viscosity when it exits the microcatheter and is no longer flowing.

One particular preferred rheological modifier is amorphous, fumed silica which is a synthetic silicon dioxide (SiO₂) that does not have the ordered structure or the health hazards of crystalline silica. Unless otherwise noted in herein, the word “silica” denotes amorphous, fumed silicon dioxide. Amorphous, fumed silica is a well-known rheology modifier used in personal care, pharmaceutical, household/industrial, and medical device applications. Typically, fumed silica is an extremely small particle with a large surface area, high purity, and a tendency to have a chain-like morphology. The fumed silica preferably, has a BET surface area of about 100 m²/g to about 700 m²/g. The term “BET” stands for stands for Brunauer, Emmett, and Teller, the three scientists who optimized the theory for measuring surface area.

Untreated, fumed silica is composed of two chemical groups: siloxane groups (Si—O—Si) and surface silanols (Si—OH) shown as isolated hydroxyls in FIG. 2.¹³

Under static conditions, the silica particles above form a “string of pearls” network via the isolated hydroxyls from separate particles, which form hydrogen bonds between the particles. Silanol hydrogen bonds (H***O) are shown in FIG. 3(A) and a model of the silica network is shown in FIG. 3(B).¹³

Amorphous, fumed silica containing surface hydroxyl groups as depicted in FIG. 2 and FIG. 3(A) is sometimes referred to herein as “amorphous, hydrophilic, fumed silica”.

When the embolic composition comprising the fumed silica is moved (e.g., by syringe-injected flow through a catheter), the silanol network created by the hydrogen bonding between the particles is broken or sheared and the fluid “thins.” This change in fluid thickness, or viscosity, is known as “shear thinning” and is reversible. That is, if the flowing fluid comes to rest, it will thicken again and repeated application of stress results in a reproducible change in viscosity.

If this reversible shear thinning occurs nearly instantaneously (less than about 5 seconds), then the fluid is referred to as “pseudo-plastic”. If the shear thinning occurs over a longer period of time, then the fluid is referred to as “thixotropic”. Thixotropic compositions are evident by measuring the shear stress (Y axis) against shear rate (X axis) at both increasing and decreasing shear rates and determining the extent of the area generated between the two curves. Compositions exhibiting pseudo-plastic behavior have little or no area between these curves whereas thixotropic compositions have measurable areas between the curves. Specifically, for the purposes of this application, compositions having an area of no more than 25,000 Pa/sec between increasing and decreasing shear rates of from 0 to 250 sec⁻¹ are deemed to be pseudo-plastic; whereas compositions having an area of greater than 25,000 Pa/sec between increasing and decreasing shear rates of from 0 to 250 sec⁻¹ are deemed to be thixotropic.

For the purposes of this application and the above definitions, all viscosity values are determined by a cone/plate instrument sold by Brookfield Engineering (Middleboro, Mass., USA) as model R/S-CPS Rheometer with RS232 interface to Windows®-based PC running Brookfield Rheocalc v2.7 software.

The term “biocompatible contrast agent” or “contrast agent” refers to a biocompatible radiopaque material capable of being monitored during injection into a mammalian subject by, for example, radiography. In the methods of this invention, the contrast agent is preferably water insoluble (i.e., has a water solubility of less than 0.01 mg/ml at 20° C.). Examples of biocompatible water-insoluble contrast agents include tantalum, tantalum oxide, and barium sulfate, each of which is commercially available in the proper form for in vivo use. Other biocompatible water-insoluble contrast agents include gold, tungsten, and platinum. Preferred biocompatible water-insoluble contrast agents are those having an average particle size of about 10 μm or less.

Water soluble contrast agents are also suitable for use herein and include, for example, lipidol, metrizamide and the like. Preferably, the biocompatible contrast agent employed does not cause a substantial adverse inflammatory reaction when employed in vivo.

The term “biocompatible polymer” refers to polymers which, in the amounts employed, are non-toxic and substantially non-immunogenic when used internally in the patient and which are substantially insoluble in the body fluid of the mammal. The biocompatible polymer can be either biodegradable or, preferably, non-biodegradable.

Biodegradable polymers are disclosed in the art. Examples of suitable biodegradable polymers include, but are not limited to, linear-chain polymers such as polylactides, polyglycolides, polycaprolactones, polyanhydrides, polyamides, polyurethanes, polyesteramides, polyorthoesters, polydioxanones, polyacetals, polyketals, polycarbonates, polyorthocarbonates, polyphosphazenes, polyhydroxybutyrates, polyhydroxyvalerates, polyalkylene oxalates, polyalkylene succinates, poly(malic acid), poly(amino acids), polyvinylpyrrolidone, polyethylene glycol, polyhydroxycellulose, polymethyl methacrylate, chitin, chitosan, and copolymers, terpolymers, and combinations thereof. Other biodegradable polymers include, for example, gelatin, collagen, etc.

Suitable non-biodegradable biocompatible polymers include, by way of example, cellulose acetates (including cellulose diacetate), ethylene vinyl alcohol copolymers (“EVOH”), hydrogels (e.g., acrylics), polyacrylonitrile, polyvinylacetate, cellulose acetate butyrate, nitrocellulose, copolymers of urethane/carbonate, copolymers of styrene/maleic acid, and mixtures thereof.

Preferably, the biocompatible polymer employed does not cause a substantial adverse inflammatory reaction when employed in vivo. The particular biocompatible polymer employed is selected relative to the viscosity of the resulting polymer solution, the solubility of the biocompatible polymer in the biocompatible solvent, and the like. For example, the selected biocompatible polymer should be soluble in the amounts employed in the selected biocompatible solvent and the resulting composition should have a viscosity suitable for in vivo delivery by the methods of this invention.

Preferred biocompatible polymers are ethylene vinyl alcohol copolymers. Other preferred polymers include cellulose acetate butyrate, cellulose diacetate, polymethyl methacrylate, polyvinyl acetate, copolymers of urethane and acrylates, and the like.

Ethylene vinyl alcohol copolymers comprise residues of both ethylene and vinyl alcohol monomers. Small amounts (e.g, less than 5 mole percent) of additional monomers can be included in the polymer structure or grafted thereon provided such additional monomers do not alter the properties of the composition. Such additional monomers include, by way of example only, maleic anhydride, styrene, propylene, acrylic acid, vinyl acetate and the like.

Ethylene vinyl alcohol copolymers are either commercially available or can be prepared by art-recognized procedures. As is apparent to one skilled in the art, with all other facts being equal, copolymers having a lower molecular weight will impart a lower viscosity to the composition as compared to higher molecular weight copolymers. Accordingly, adjustment of the viscosity of the composition as necessary for catheter delivery can be readily achieved by merely adjusting the molecular weight of the copolymer composition.

As is also apparent, the ratio of ethylene to vinyl alcohol in the copolymer affects the overall hydrophobicity/hydrophilicity of the composition which, in turn, affects the relative water solubility/insolubility of the composition as well as the rate of precipitation of the copolymer in an aqueous environment (e.g., blood or tissue). In a particularly preferred embodiment, the copolymers employed herein comprise a mole percent of ethylene of from about 25 to about 60 and a mole percent of vinyl alcohol of from about 40 to about 75. These compositions provide for requisite precipitation rates suitable for use in the methods described therein.

The term “biocompatible solvent” refers to an organic material liquid at least at body temperature of the mammal in which the biocompatible polymer is soluble and, in the amounts used, is substantially non-toxic. Suitable biocompatible solvents include, by way of example, ethyl lactate, dimethylsulfoxide (“DMSO”), analogues/homologues of dimethylsulfoxide, ethanol, acetone, and the like. Aqueous mixtures with the biocompatible solvent can also be employed, provided that the amount of water employed is sufficiently small that the dissolved polymer precipitates upon contact with blood or other bodily fluid. Preferably, the biocompatible solvent is dimethylsulfoxide.

The term “biocompatible prepolymer” refers to materials which polymerize in situ to form a polymer which polymer, in the amounts employed, is non-toxic, chemically inert, and substantially non-immunogenic when used internally in the patient and which is substantially insoluble in blood. Suitable biocompatible prepolymers include, by way of example, cyanoacrylates, hydroxyethyl methacrylate, silicon prepolymers, and the like. The prepolymer can either be a monomer or a reactive oligomer, or a two component prepolymer such as those disclosed by Porter.¹⁴ Preferably, the biocompatible prepolymer forms a polymer which is also non-inflammatory when employed in vivo.

The term “bridging molecule” means a substance which facilitates aggregation under static conditions of the rheological modifier in the composition. Suitable bridging molecules include glycols [HO—(C₂-C₆ alkylene)-OH], and derivatives of glycol, such as [HO—(C₂-C₆ alkylene-O]ₙH polymers where n is greater than one and is preferably from about 7 to about 333.

The term “embolizing” refers to a process wherein a material is injected into a blood vessel which, in the case of, for example, aneurysms, fills or plugs the aneurysmal sac and/or encourages clot formation so that blood flow into the aneurysm ceases. In the case of AVMs, a plug or clot is formed to control/reroute blood flow to permit proper tissue perfusion. In the case of a vascular site, the vascular site is filled to prevent blood flow there through. Embolization of the blood vessel is important in preventing and/or controlling bleeding due to lesions (e.g., organ bleeding, gastrointestinal bleeding, vascular bleeding, and bleeding associated with an aneurysm). In addition, embolization can be used to ablate diseased tissue (e.g., tumors, etc.) by cutting off the diseased tissue's blood supply.

The term “encapsulation” as used relative to the contrast agent being encapsulated in the polymer precipitate does not infer any physical entrapment of the contrast agent within the precipitate, much as a capsule encapsulates a medicament. Rather, this term is used to mean that an integral, coherent precipitate forms which does not separate into individual components.

“cP” as used herein refers to centipoise, which is related to the centistokes by the material's density.

The term “irradiation” as it relates to sterilization refers to all forms of irradiation capable of effecting sterilization including gamma irradiation, e-beam irradiation, visible light irradiation, UV irradiation and the like. Examples of suitable forms of irradiation are provided by Grieb, et al., U.S. Pat. No. 6,696,060, which is incorporated herein by reference in its entirety.

“Static conditions” as used herein means that less than about 1 s⁻¹ of shear stress is applied.

“Surfactants” are those substances which enhance flow and/or aid dispersion by reducing surface tension when dissolved in water or water solutions, or that reduce interfacial tension between two liquids, or between a liquid and a solid. Surfactants also impede the interaction between the rheological modifier and other components of the system. This allows a more fully developed rheological modified system. Surfactants may be anionic, cationic, and nonionic. Surfactants include detergents, wetting agents, and emulsifiers. Suitable cationic surfactants include organic amines and organic ammonium chlorides (e.g., N-tallow trimethylene diamine diolealate and N-alkyl trimethyl ammonium chloride) and the like. Suitable anionic surfactants include, by way of example sulfosuccinates, carboxylic acids, alkyl sulfonates, octoates, oleates, stearates, and the like. Suitable nonionic surfactants, include by way of example, bridging molecules discussed above, tritons, tweens, spans and the like. Polyfunctional additives such as glycerin and various glycols may be added. The adjustment of pH by the addition of potassium or sodium hydroxide ionizes silanols and alters the composition's rheology.

The term “biocompatible plasticizer” refers to any material which is soluble or dispersible in the embolic composition, which increases the flexibility of the resulting polymer mass formed in vivo, and which, in the amounts employed, is biocompatible as measured by the lack of moderate to severe tissue irritation. Suitable plasticizers are well known in the art and include those disclosed in U.S. Pat. Nos. 2,784,127 and 4,444,933. The disclosures of both of these patents are incorporated herein by reference in their entirety. Specific plasticizers include, by way of example only, acetyl tri-n-butyl citrate (preferably about 20 weight percent or less), acetyl trihexyl citrate (preferably about 20 weight percent or less) butyl benzyl phthalate, dibutyl phthalate, dioctylphthalate, n-butyryl tri-n-hexyl citrate, diethylene glycol dibenzoate (preferably about 20 weight percent or less) and the like. The particular biocompatible plasticizer employed is not critical and preferred plasticizers include dioctylphthalate and acetyl tri-n-butyl citrate.

The term “initial fluence” of E-beam radiation refers to the fluence of this beam immediately after release from the E-beam accelerator. As is well known, the fluence of an E-beam will be reduced the further it travels from the source.

The term “sanitizing agent” refers to any agent compatible with the packaging elements which, when contacted with these elements, sanitizes the package by reducing bioburden thereon. Preferably, bioburden is reduced to levels of less than about 10 colony forming units (CFU) on individual packaging elements and more preferably less than about 3 CFUs. Preferred sanitizing agents include, for example, heat, plasma and ethylene oxide. Other suitable sanitizing agents are well known in the art.

Compositions and Methods of Preparation these Compositions prior to Sterilization

The embolic compositions sterilized as per this invention are prepared by the methods as set forth below in order to achieve the desired properties.

I. Embolic Compositions Comprising Hydroxyl-Containing Rheological Modifiers

A. Polymeric Embolic Compositions.

In a first embodiment, the embolic compositions comprise a biocompatible polymer, a biocompatible solvent, a hydroxyl-containing rheological modifier and optionally a contrast agent. These compositions are prepared by adding sufficient amounts of a biocompatible polymer, an optional biocompatible contrast agent, a biocompatible solvent, and the rheological modifier to achieve the effective concentration for the rheologically-modified composition.

The embolic polymer compositions employed herein are prepared by the methods set forth below, whereby each of the components is added and the resulting embolic composition is mixed until it is substantially homogeneous. Generally, the embolic compositions can be prepared by adding sufficient amounts of the biocompatible polymer to the biocompatible solvent to achieve the effective concentration for the embolic composition. If necessary, gentle heating and stirring can be used to effect dissolution of the biocompatible polymer into the biocompatible solvent, e.g., 12 hours at 55° C. Excessive heating should not be used in order to prevent evaporation of the solvent and degradation of the polymer component(s).

Each of the polymers recited herein is commercially available but can also be prepared by methods known in the art. For example, polymers are typically prepared by conventional techniques such as radical, thermal, UV, gamma irradiation, or electron beam induced polymerization employing, as necessary, a polymerization catalyst or initiator to provide for the polymer composition.

The specific manner of polymerization is not critical and the polymerization techniques employed do not form a part of this invention.

In order to maintain solubility in the biocompatible solvent, the polymers described herein are preferably not cross-linked.

When employed, sufficient amounts of the contrast agent are then added to the polymer solution to achieve the effective concentration for the composition. When the contrast agent is water insoluble, the particle size of water insoluble contrast agent is preferably maintained at about 10 μm or less and more preferably at from about 1 to about 5 μm (e.g., an average size of about 2 μm) so as to enhance the formation of a homogeneous suspension.

After addition of the polymer and optionally the contrast agent to the solvent, the hydroxyl-containing rheological modifier is added under ambient conditions, preferably under inert atmosphere, for example, an argon atmosphere. If a particulate rheological modifier is used, the composition is initially stirred at low RPM (less than about 1000 RPM) to wet the surface of the rheological modifier. Once wetted, the stir rate is increased to a peripheral tip speed of from about 5 m/sec to about 26.5 m/sec. The tip speed should be maintained until no granular material is evidenced in composition. When soluble rheological modifiers are used, the composition need not be stirred at low RPM and are easily added to the composition.

The initial viscosity of the composition is controlled at least in part by the amount of polymer employed and/or its molecular weight. For example, high-viscosity compositions which employ low concentrations of polymer can be achieved by the use of very high molecular weight biocompatible polymers (e.g., those with an average molecular weight greater than 250,000). In the alternative, an high-viscosity composition may be achieved with the use a low molecular weight polymer at a high concentration. Such factors are well known in the art and modification of these parameters will be well within the abilities of one of skill in the art.

The viscosity of the composition is then modified by the addition of the hydroxyl-containing rheological modifier. The addition of the hydroxyl-containing rheological modifier provides a decrease in the viscosity under shear stress and an increase in the viscosity and/or yield stress under static conditions.

The rheologically-modified composition is stirred as necessary to achieve homogeneity of the composition. Preferably, mixing/stirring of the composition is conducted under an anhydrous atmosphere at ambient pressure.

In one preferred embodiment the rheologically-modified composition is first mixed in a mixer at a low shear (stir) rate wherein the peripheral tip speed of the mixing blades is less than about 10 m/s, and more preferably about 1 m/s.

In a particularly preferred embodiment, high shear dispersion (HSD) of the silica in the embolic composition is achieved by a peripheral tip speed (PTS) of from about 9 m/sec to about 20 m/sec. The PTS measures how much circumferential distance the mixing blade travels per unit time and can be calculated from the revolutions per minute (rpm) and the known dimensions of the mixing blade per the following formula:

PTS(m/sec)=(RPM)(3.14)(Diameter of the Blade in m)(1 minute/60 sec)

Preferably, the blade should be about 0.5 to 1 blade diameter above the bottom of the mixing vessel. Preferably, the diameter of the mixing vessel should be 2 to 3 times the diameter of the mixing blade.

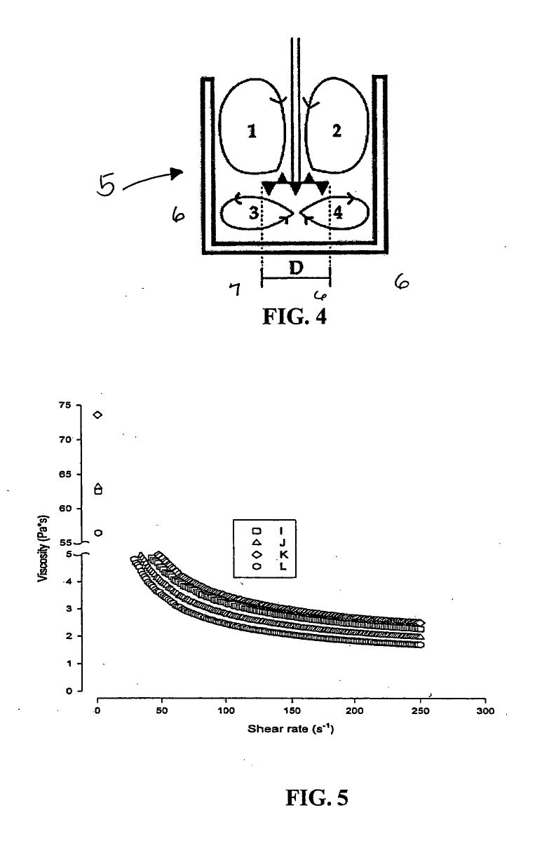

FIG. 4 illustrates a prototypical mixing blade and mixing vessel. Specifically, mixing vessel 5 comprises a vessel chamber defined by walls 6 and a mixing blade 7 having a blade diameter D. Upon rotation of mixing blade 7, the fluid inside the vessel chamber will be mixed in a manner to define 4 separate vortices denoted by 1, 2, 3 and 4.

If desired, the rheologically-modified composition can be degassed either prior to or after the rheological modifier is added. The degassing can be performed by any conventional degassing technique, e.g., vacuum treatment. In one suitable process, the post-mix material is placed under vacuum for degassing for a prolonged period (e.g., about 40 millibar for at least about 12 hours).

In another alternative embodiment, the rheological modifier can be added to the biocompatible solvent, e.g., DMSO, before the mixing of the overall composition. For example, in one embodiment, the solvent could be divided into two portions wherein one portion equaling about ⅓ of the total solvent is added to the polymer. Meanwhile, the remaining ⅔ is blended with the rheological modifier. The rheological modifier and the biocompatible solvent is blended to a homogenous composition and then blended with the composition to form the rheologically-modified composition.

The method for manufacturing a rheologically-modified composition as set forth above is only one permutation of the methods for manufacturing the rheologically-modified composition. It can be appreciated that the rheological modifier could be initially blended with the polymer and then added to the solvent and contrast agent, or any combination, including but not limited to adding the rheological modifier to the polymer, solvent or contrast agent or any combination thereof.

Scheme 1 below illustrates protocols for preparing each of the preferred components ultimately employed in the preferred embolic compositions prepared as per the methods described above.

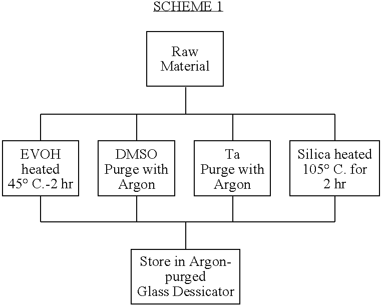

It is understood, of course, that these preferred components are merely representative of other components and that other particulate agents other than silica can be treated in a similar manner as silica in Scheme 1. Likewise, biocompatible polymers other than ethylene vinyl alcohol copolymer (EVOH), biocompatible solvents other than DMSO and water insoluble contrast agents other than tantalum (Ta) can be treated in a similar manner to EVOH, DMSO and Ta respectively.

These protocols minimize the presence of water in each of the starting (raw) materials. Embolic compositions not containing either the rheological modifier and/or the water insoluble contrast agent follow similar protocols with the exception that one or both of these components are not treated. Similarly, when a soluble rheological modifier is employed, the protocol set forth for EVOH can be employed for such soluble rheological modifiers. Still further, when a water soluble contrast agent is employed and it is a liquid, it is treated in a manner similar to DMSO.

When soluble rheological modifiers are used, the composition need not be stirred at low RPM as such soluble modifiers are easily added to the composition. Regardless of the type of rheological modifier used, the rheologically-modified composition is generally stirred from about 2.5 minutes to about 20 minutes, more preferably from 2.5 to 10 minutes. In one embodiment, a 10 minute stir is employed and the stirring is interrupted after 5 minutes to scrape material from the vessel chamber walls 6 down into the main mix. Afterwards, mixing was resumed.

Preferably, the composition will comprise from about 1:1 to about 2:1 weight of biocompatible polymer to the rheological modifier, and even more preferably from about 1.2:1 to about 1.4:1 weight of biocompatible polymer to the rheological modifier. Insofar as the particulate rheological modifier is not soluble in the biocompatible solvent, stirring is employed to effect homogeneity of the resulting suspension.

Optionally, coated rheological modifier, such as coated silica may be employed as a particulate rheological modifier in the rheologically-modified composition. The coating on the rheological modifier is preferably biocompatible and preferably coated in a polymer that is insoluble in both water and biocompatible solvent, e.g. DMSO. The coating is also preferably non-toxic or biocompatible.

The rheological modified composition is stirred as necessary to achieve homogeneity of the composition. Preferably, mixing/stirring of the composition is conducted under an anhydrous atmosphere at ambient pressure.

Preferably compositions meeting the above criteria, i.e., higher viscosity, would be used in the treatment of aneurysms, arteriovenous fistulae and the like. When treating such conditions, it is desirable that the composition have proximal occlusion at the vascular site. In other words, it is desirable that the composition act like a plug.

At times percentages are expressed in conventional relative weight percent of the final product and at other times the percentages are expressed as illustrated below.

A preferred composition has a range of biocompatible polymer (weight/volume solvent) to fumed silica (weight/final weight) of from at least about 2.6 to 1 to about 3.6 to 1. More preferably, the ratio is about 3.0 to 1 to about 3.2 to 1. Even more preferably, the ratio is about 3.1 to 1.

This ratio is rather unconventional but is an artifact of the way these compositions are assembled. In assembly, a weight in grams of biocompatible polymer is dispersed/dissolved in 100 ml of solvent. A weight of contrast agent is added and a weight addition of fumed silica is incorporated. The ratio of polymer as the initial weight in grams (g) per volume of solvent in milliliters (ml) is then divided by the weight of silica in the weight of final product in percent.

For example when 19 g of EVOH are dispersed in 100 ml DMSO (D=1.10, 100 ml=110 g) and tantalum is added to yield 38% weight to weight of the final product and silica is added to yield 6.175% weight to final weight of the product, the ratio is 19/6.175 or 3.1 to 1.

Alternatively, the amounts of these polymer and modifier materials can be defined by a more conventional ratio of the percent by weight biocompatible polymer to percent weight of fumed silica, both based on the final weight of the composition. This ratio ranges from about 1.4 to 1 to about 0.9 to 1, especially 1.3 to 1 to 1.0 to 1 and preferably 1.1 to 1.

Based on in vitro and in vivo testing, four rheologically modified compositions employing the following ratios of amorphous fumed silica (silica) as the rheological modifier and ethylene vinyl alcohol copolymer (EVOH) as the biocompatible polymer were determined to provide overall beneficial properties both during delivery and upon ejection into aneurysmal sac:

Formula I: 6.25% EH5 silica (wt/final wt) and 7.83% EVOH (wt/final wt)

Formula J: 6.28% EH5 silica (wt/final wt) and 7.41% EVOH (wt/final wt)

Formula K: 6.175% EH5 silica (wt/final wt) and 8.21% EVOH (wt/final wt)

Formula L: 6.38% EH5 silica (wt/final wt) and 6.98% EVOH (wt/final wt)

Each of these formulas contains approximately 38 wt/final wt tantalum with the balance being DMSO.

FIG. 5 illustrates the relationship non-Newtonian viscosity of these compositions over a range of shear rates. Specifically, FIG. 5 illustrates that these compositions exhibit nominal viscosities at high shear rates and rapidly increasing viscosities at low shear rates.

For optimum control at the neck of the aneurysm, polymeric embolic compositions comprising EVOH and amorphous, hydrophilic fumed silica preferably have a weight percent of fumed silica ranging from 5 to 7 percent based on the total weight of the composition and more preferably have approximately, 6.26±0.01 weight percent. These compositions preferably have a weight percent of EVOH of from 6 to 9 percent based on the total weight of the composition and more preferably have about 8.20±0.03 weight percent.

Particularly preferred rheologically-modified compositions, both in the higher and lower viscosity ranges discussed above, are shown in the table below. All of the % values are % weight based on the weight of the final product:

| | | Particularly |
|---|---|---|
| Component | Preferred | preferred |
| Biocompatible Polymer | 1 to 12% | 3 to 9% |
| Contrast Agent | 20 to 55% | 37 to 40% |
| Fumed Silica | 1 to 12% | 3 to 10% |

Further explanation of silica-containing, embolic compositions useful in this invention, is provided in U.S. patent application Ser. No. __/___.___, entitled FUMED SILICA EMBOLIC COMPOSITIONS, filed Feb. 26, 2004 with Attorney Docket No. 55492-20093.00 which application is incorporated herein by reference in its entirety.

B. Prepolymer Embolic Compositions.

In a second embodiment, the embolic compositions comprise a biocompatible prepolymer, an optional biocompatible solvent, a hydroxyl-containing rheological modifier and optionally a contrast agent. These compositions are prepared by adding sufficient amounts of a biocompatible prepolymer, an optional biocompatible contrast agent, an optional biocompatible solvent, and a hydroxyl-containing rheological modifier to achieve the effective concentration for the rheologically-modified embolic composition.

As per Scheme 1 above, each of the hydroxyl-containing rheological modifier, the optional biocompatible solvent and the optional contrast agent is pre-treated prior to addition. Liquid prepolymers are also pretreated in a manner similar to that of the biocompatible solvent.

Generally, the viscosity of an unmodified composition, i.e., a composition comprising a biocompatible prepolymer, an optional biocompatible solvent and an optional contrast agent, is controlled by the prepolymer employed and the unmodified composition exhibits a Newtonian viscosity profile which, due to the molecular weight of the prepolymer is typically less than 500 cP at 37° C.

However, in rheologically-modified compositions, the viscosity of this composition is also controlled by the rheological modifier employed, for example, fumed silica, and these compositions exhibit a non-Newtonian viscosity profile.

The embolic prepolymer compositions employed herein are prepared by the methods set forth below, whereby each of the components is added and the resulting embolic composition is mixed until it is substantially homogeneous. Generally, if these embolic compositions employ a biocompatible solvent, these compositions can be prepared by adding sufficient amounts of the biocompatible prepolymer to the biocompatible solvent to achieve the effective concentration for the embolic composition.

When employed, sufficient amounts of the contrast agent are then added to the prepolymer to achieve the effective concentration for the composition. When the contrast agent is water insoluble, the particle size of water insoluble contrast agent is preferably maintained at about 10 μm or less and more preferably at from about 1 to about 5 μm (e.g., an average size of about 2 μm) so as to enhance the formation of a homogeneous suspension.

Afterwards, the rheological modifier is added under ambient conditions, preferably under inert atmosphere, for example, an argon atmosphere. If a particulate rheological modifier is used, the composition is initially stirred at low RPM (less than about 1000 RPM) to wet the surface of the rheological modifier. Once wetted, the stir rate is increased to a peripheral tip speed of from about 5 m/sec to about 26.5 m/sec. The tip speed should be maintained until no granular material is evidenced in composition. When soluble rheological modifiers are used, the composition need not be stirred at low RPM and are easily added to the composition.

In a particularly preferred embodiment, high shear dispersion (HSD) of the silica in the embolic composition is achieved by a peripheral tip speed (PTS) of from about 9 m/sec to about 20 m/sec. The PTS measures how much circumferential distance the mixing blade travels per unit time and can be calculated from the revolutions per minute (rpm) and the known dimensions of the mixing blade per the following formula:

PTS (m/sec)=(RPM)(3.14)(Diameter of the Blade in m)(1 minute/60 sec)

Preferably, the blade should be about 0.5 to 1 blade diameter above the bottom of the mixing vessel. Preferably, the diameter of the mixing vessel should be 2 to 3 times the diameter of the mixing blade.

As before, FIG. 4 illustrates a prototypical mixing blade and mixing vessel. Specifically, mixing vessel 5 comprises a vessel chamber defined by walls 6 and a mixing blade 7 having a blade diameter D. Upon rotation of mixing blade 7, the fluid inside the vessel chamber will be mixed in a manner to define 4 separate vortices denoted by 1, 2, 3 and 4.

The preparation of starting materials proceeds as described above in Scheme 1 except that the prepolymer is not heated but, rather, is purged with argon. When a soluble rheological modifier is employed, the protocol set forth for EVOH in Scheme 1 can be employed for such soluble rheological modifiers. Still further, when a water soluble contrast agent is employed and it is a liquid, it is treated in a manner similar to DMSO.

When soluble rheological modifiers are used, the composition need not be stirred at low RPM as such soluble modifiers are easily added to the composition. Regardless of the type of rheological modifier used, the rheologically-modified composition is generally stirred from about 2.5 minutes to about 20 minutes, more preferably from 2.5 to 10 minutes. In one embodiment, a 10 minute stir is employed and the stirring is interrupted after 5 minutes to scrape material from the vessel chamber walls 6 down into the main mix. Afterwards, mixing was resumed.

Preferably, the composition will comprise from about 1:1 to about 2:1 weight of biocompatible prepolymer to the rheological modifier, and even more preferably from about 1.2:1 to about 1.4:1 weight of biocompatible prepolymer to the rheological modifier. Insofar as the particulate rheological modifier is not soluble in the biocompatible solvent, stirring is employed to effect homogeneity of the resulting suspension.

Optionally, coated rheological modifier, such as coated silica may be employed as a particulate rheological modifier in the prepolymeric embolic composition. The coating on the rheological modifier is preferably biocompatible and preferably coated in a polymer that is insoluble in both water and biocompatible solvent, e.g. DMSO. The coating is also preferably nontoxic or biocompatible.

The prepolymeric embolic composition is stirred as necessary to achieve homogeneity of the composition. Preferably, mixing/stirring of the composition is conducted under an anhydrous atmosphere at ambient pressure.

It is preferred that the mixing of the composition under high shear conditions is maintained for a period of time sufficient such that the resulting composition has a viscosity under static conditions of at least 4000 cP at 37° C. and a viscosity under shear conditions of less than 2000 cP at 37° C.

A particularly preferred prepolymeric composition comprises a solution of about 3 to about 12 weight percent of biocompatible prepolymer, 20 to about 55 weight percent of a contrast agent, preferably about 37 to about 40 weight of the contrast agent, and about 3 to about 12 percent fumed silica. All of the above percentage values are based on the total weight of composition.

If desired, the prepolymeric embolic composition can be degassed either prior to or after the rheological modifier is added. The degassing can be performed by any conventional degassing technique, e.g., vacuum treatment. Preferably, the post-mix material is placed under a vacuum for degassing, (e.g., about 40 millibar for at least about 12 hours).

In another alternative embodiment, the rheological modifier can be added to the biocompatible solvent, e.g., DMSO, before the mixing of the composition. For example, in one embodiment, the solvent could be divided into two portions wherein one portion equaling about one-third of the total solvent is added to the prepolymer. Meanwhile, the remaining two-thirds is blended with the rheological modifier. The rheological modifier and the biocompatible solvent is blended to a homogenous composition and then blended with the prepolymer to form the rheologically-modified composition.

The method for manufacturing a prepolymeric composition as set forth above is only one permutation of the methods for manufacturing these compositions. It can be appreciated that the rheological modifier could be initially blended with the prepolymer and then added to the solvent and contrast agent, or any combination, including but not limited to adding the rheological modifier to the polymer, solvent or contrast agent or any combination thereof.

C. Other Components

Surfactants can be optionally employed in the embolic compositions described herein. When employed, surfactants maintain dispersion of the rheological modifier and/or the contrast agent. Surfactants also impede the interaction between the rheological modifier and other components of the system. This allows for more fully developed rheologically-modified system.

When surfactants are employed, a preferred biocompatible rheologically-modified composition comprises from about 1 to about 12 weight percent of biocompatible polymer, from about 20 to about 55 weight percent of a contrast agent, preferably about 37 to about 40 weight percent of contrast agent, from about 1 to about 12 percent of the rheological modifier, all based upon total weight of composition and from about 10 to about 20 weight percent of surfactant, based upon the weight of silica and the remaining weight percent biocompatible solvent.

Bridging molecules may also be employed in the biocompatible rheologically-modified compositions of the instant invention. When employed, bridging molecules act as a dispersion modifier for the rheological modifiers.

When bridging molecules are employed, a preferred biocompatible rheologically-modified composition comprises from about 1 to about 12 weight percent of biocompatible polymer, from about 20 to about 55 weight percent of a contrast agent, preferably about 37 to about 40 weight percent of contrast agent, from about 1 to about 12 percent rheological modifier, all based upon total weight of composition and from about 10 to about 30 weight percent of bridging molecule, based upon the weight of silica and the remaining weight percent biocompatible solvent.

A biocompatible plasticizer can also be added to the composition. When employed, the plasticizer imparts flexibility to the solidified composition and prevents brittleness of the solidified polymer. The desired plasticizer is compatible with the both the embolic composition and the solid mass formed in vivo and imparts properties such as flexibility, elasticity, and minimal catheter adhesivity, to the solidified composition. Preferably, the plasticizer employed is either dioctyl phthalate or tri-n-butyl acetyl citrate. The amount of plasticizer employed is sufficient to impart flexibility to the resulting precipitate formed in vivo while maintaining the embolic properties of the composition. Preferably, the plasticizer is employed from about 10 to 20 weight percent based on the weight of the biocompatible polymer or prepolymer employed.

It is also contemplated that sensitizers can be added to the embolic composition to enhance the sterilizing effect of the irradiation employed. Such sensitizers include substances that selectively targets viral, bacterial, prion and/or parasitic contaminants, rendering them more sensitive to inactivation by radiation, therefore permitting the use of a lower rate or dose of radiation and/or a shorter time of irradiation than in the absence of the sensitizer. Illustrative examples of suitable sensitizers include, but are not limited to, the following: psoralen and its derivatives and analogs (including 3-carboethoxy psoralens); angelicins, khellins and coumarins which contain a halogen substituent and a water solubilization moiety, such as quaternary ammonium ion or phosphonium ion; nucleic acid binding compounds; brominated hematoporphyrin; phthalocyanines; purpurins; porphorins; halogenated or metal atom-substituted derivatives of dihematoporphyrin esters, hematoporphyrin derivatives, benzoporphyrin derivatives, hydrodibenzoporphyrin dimaleimade, hydrodibenzoporphyrin, dicyano disulfone, tetracarbethoxy hydrodibenzoporphyrin, and tetracarbethoxy hydrodibenzoporphyrin dipropionamide; doxorubicin and daunomycin, which may be modified with halogens or metal atoms; netropsin; BD peptide, S2 peptide; S-303 (ALE compound); dyes, such as hypericin, methylene blue, eosin, fluoresceins (and their derivatives), flavins, merocyanine 540; photoactive compounds, such as bergapten; and SE peptide.

Sterilization

Sterilization of the embolic compositions prepared as above preferably proceeds via irradiation techniques.

E-Beam Sterilization

In one embodiment, sterilization proceeds via e-beam sterilization techniques. In this preferred embodiment, the embolic composition is first packaged into a suitable container which is preferably air-tight and moisture resistant. Such containers include, for example, aluminum tubes, glass, polyalkylene based polymers such as polypropylene or polyethylene, metal foils, and the like.

In a preferred embodiment, the packaging element comprises aluminum tubes having a capacity of from about 1 to 20 g of embolic composition.

The packaging element is filled to the desired level with the embolic composition using any of several well known filling methods and the particular filling method is not critical to this invention and does not form part of the claimed invention. A preferred method for filling the packaging element comprises syringe transfer of the embolic composition using a piston pump.

Once filled, the packaging elements are preferably sealed, again by conventional means such as crimping. If necessary, the sealing means can include auxiliary sealing means. For example, an ampoule comprising a screw cap sealing means can be further sealed by placement of a removable polymer coated metal foil (e.g., polyethylene coated foil) over the mouth of the ampoule to which the screw cap overlays. Again, any conventional sealing means can be used as the sealing means does not form any part of this invention.

The packaging element, whether an individual element or individual elements combined into larger packaging elements, is subjected to E-beam sterilization. The E-beam generator is any of the conventional and well known generators of high energy electrons which are commercially available for this purpose. In addition, the E-beam radiation employed is preferably maintained at an initial fluence of at least 2 μCurie/cm², preferably at least 4 μCurie/cm², more preferably at least 8 μCurie/cm² and even more preferably 10 μCurie/cm². Preferably the E-beam radiation employed has an initial fluence of from about 2 to about 50 μCurie/cm².

The dose of E-beam radiation employed is one sufficient to sterilize the packaging element as well as its contents. In a preferred embodiment, the E-beam dosage is preferably from about 2 to 75 kGray and more preferably from about 15 to about 30 kgray with the specific dosage being selected relative to the density of material being subjected to E-beam radiation as well as the amount of bioburden estimated to be therein. Such factors are well within the skill of the art. Upon completion of the sterilization process, the sterilized product is ready for shipment to the ultimate user or can be packaged into larger shipping elements.

E-beam sterilization is preferably conducted at ambient atmospheric conditions such as a temperature of from about 0° C. to about 40° C., although initially, the temperature is preferably room temperature. It is contemplated that a slight temperature increase may occur during irradiation. The exposure time of the product to the E-beam radiation is dependent on the fluence of the radiation employed and the dosage required which is well within the skill of the art. Preferably, exposure of the product to the E-beam is less than 60 seconds.

In an optional embodiment, sterilization of the embolic composition is facilitated by employing steps to reduce biocontamination of the packaging element and/or the embolic composition prior to E-beam sterilization. For example, the packaging element can be contacted with compatible sterilization or sanitization conditions prior to fill to reduce bioburden thereon. Since these sterilization or sanitization conditions are employed prior to incorporation of the embolic composition, sterilization or sanitization conditions which are compatible with the packaging but would be otherwise incompatible with embolic composition can be used including, for example, steam sterilization, heat sterilization, etc.

Using such steps prior to irradiation with E-beams effectively reduces the E-beam dosage necessary to sterilize the composition.

Gamma Irradiation

Alternatively, gamma irradiation can be employed to effect sterilization of the embolic composition. When employed, the dose of gamma irradiation used to sterilize the embolic composition is similar to that employed with e-beam sterilization techniques. That is to say that the dose is from about preferably from about 2 to 75 kGray and more preferably from about 15 to about 30 kGray with the specific dosage being selected relative to the quantity of material to be sterilized as well as the amount of bioburden estimated to be therein. Such factors are well within the skill of the art. Upon completion of the sterilization process, the sterilized product is ready for shipment to the ultimate user or can be packaged into larger shipping elements.

The embolic composition is irradiated for a time effective for the inactivation of one or more biological contaminants contained therein. Combined with irradiation rate, the appropriate irradiation time can be readily determined by one skilled in the art.

Optionally, an effective amount of at least one sensitizing compound may be added to the composition to irradiation to enhance the anti-microbial effect of the irradiation.

Gamma irradiation is preferably conducted under ambient conditions such as a temperature of from about 0° C. to about 40° C., although initially, the temperature is preferably room temperature. It is contemplated that a slight temperature increase may occur during irradiation.

In an optional embodiment, sterilization of the embolic composition is facilitated by employing steps to reduce biocontamination of the packaging element and/or the embolic composition prior to sterilization via gamma irradiation. For example, the packaging element can be contacted with compatible sterilization or sanitization conditions prior to fill to reduce bioburden thereon.

Since these sterilization or sanitization conditions are employed prior to incorporation of the embolic composition, sterilization or sanitization conditions which are compatible with the packaging but would be otherwise incompatible with embolic composition can be used including, for example, steam sterilization, heat sterilization, etc.

Using such steps prior to gamma irradiation reduces the dosage necessary to sterilize the composition.

In addition to the above, other irradiation methods for sterilizing the embolic composition include those described by Grieb, et al., U.S. Pat. No. 6,696,060, which is incorporated herein by reference in its entirety.

Sterilization of Reduced Hydroxyl-Content Rheological Modifiers

As noted previously and again without being limited to any theory, it is postulated that during heat sterilization, dehydration of the hydroxyl groups of the surface hydroxyl groups of hydroxyl-containing rheological modifiers and/or hydroxyl groups of the biocompatible polymer results in covalent linkages rather than the reversible hydrogen bond linkages that existed in the non-sterile material. It is further postulated that this new structure is more viscous and requires more shear stress to flow.

Accordingly, sterilization of embolic compositions comprising a sufficient amount of one or more hydroxyl-containing rheological modifiers to effect shear-thinning and pseudo-plastic behavior to the composition, including heat sterilization, can be facilitated by reducing the amount of surface hydroxyl functionality on the hydroxyl-containing rheological modifier(s). When so reduced, the sterilized composition exhibits a minimal increase in its thixotropic behavior as compared to the composition prior to sterilization which is characterized by an area between the two curves measuring shear stress at increasing and decreasing shear rates measured at from 0 to 250 s⁻¹ of no more than about 25,000 Pa/sec.

Specifically, in this aspect of the invention, the surface hydroxyl groups of the hydroxyl-containing rheological modifier are reduced by at least about 25% by conversion to non-hydroxyl groups. Preferably, at least about 50% of the hydroxyl groups are removed from the surface of the hydroxyl-containing rheological modifier by conversion to non-hydroxyl groups; more preferably, 90% and, still more preferably, at least 98% of the surface hydroxyl groups are removed. In a particularly preferred embodiment, products free of surface hydroxyl groups are employed.

When the hydroxyl-containing rheological modifier is amorphous, hydrophilic fumed silica, then surface hydroxyl groups can be removed by conversion from silanol groups to siloxane groups by conventional means well known in the art.

Silica which has been surface treated to provide for essentially no surface silanol groups is also commercially available from Cabot Corp., Illinois, USA under the tradename, TS-720.

Surface hydroxyl groups on soluble hydroxyl-containing rheological modifiers such as poly(2-hydroxyethyl-acrylates), copolymers of ethylene and maleic acid, polyvinylalcohol, hydroxypropylcellulose, hydroxypropylmethylcellulose, carboxymethylcellulose, sodium hydroxyethylcellulose, hydroxyethylcellulose, methylcellulose, poly(2-hydroxy-ethylmethacrylates), and the like can be converted into non-hydroxyl functionality by conventional means well known in the art. For example, the hydroxyl groups can be acylated and products such as triacetyl methylcellulose are commercially available. Alternatively, conventional reaction of a hydroxyl group with an isocyanate compound provides for non-hydroxylcarbamate groups. Still further, oxidation of the hydroxyl group to the corresponding ketone or aldehyde can be accomplished under conventional conditions.

Sterilization of the embolic compositions comprising these modified hydroxyl-containing rheological modifiers can proceed via irradiation as noted above or, alternatively, by heat sterilization. In a preferred embodiment, sterilization proceeds via dry heating the composition under conditions sufficient to sterilize the composition preferably at about 130° C. ±5° C. for approximately 90 minutes.

Example 6 below illustrates the extent of thixotropy on sterilized embolic compositions comprising partially treated silica (approximately 50% of the surface silanol groups have been reacted) and completely treated silica as the rheological modifier as compared to a non-sterile embolic composition comprising untreated silica as the rheological modifier. Specifically, the non-sterile embolic composition comprising untreated silica as the rheological modifier shows excellent pseudo-plastic properties. Contrarily, use of partially treated silica as the rheological modifier provides for a sterilized embolic composition exhibiting a decreased pseudo-plastic behavior (area=approximately 11,000 Pa/sec) but nevertheless sufficiently pseudo-plastic to be useful in this invention. The use of completely treated silica as the rheological modifier provides for a sterilized embolic composition exhibiting even better pseudo-plastic behavior as compared to both other compositions.

Utility

The compositions and methods described herein are useful in embolizing mammalian blood vessels and thus can be used to prevent/control bleeding (e.g., organ bleeding, gastrointestinal bleeding, vascular bleeding, bleeding associated with an aneurysm or an AVM) or to ablate diseased tissue (e.g., tumors, etc.). Accordingly, the invention finds use in human and other mammalian subjects requiring embolization of blood vessels. These composition can also be used in tissue bulking including sphincter bulking, peri-uretheral tissue bulking, soft-tissue augmentation as described in U.S. Pat. Nos. 6,231,613; 6,238,335; 6,595,910; and 6,569,417; and the like each of which is incorporated herein by reference in their entirety.

Methods for bulking tissue are preferably accomplished by delivering via a delivery device at the tissue site to be bulked a composition of this invention. Such methods preferably comprise inserting the delivery device into the selected tissue, delivering via the device a composition comprising a non-reactive biocompatible substance, a sufficient amount of a rheological modifier to permit the composition to exhibit thixotropic behavior, optionally a contrast agent and/or a biocompatible liquid that is miscible in blood or other body fluid under conditions wherein a mass is formed which bulks the tissue.

Suitable tissue sites for bulking include the suburethral tissue, the periurethreal tissue, soft tissue and sphincters such as the esophageal sphincter.

Suitable delivery devices includes syringes, catheters, and the like.

It is contemplated that the compositions can be employed as a carrier for a compatible, pharmaceutically-active compound wherein this compound is delivered in vivo for subsequent release. Such compounds include by way of example only antibiotics, anti-inflammatory agents, chemotherapeutic agents, anti-angiogenic agent, radioactive agents, growth factors and the like.

The following examples are set forth to illustrate the claimed invention and are not to be construed as a limitation thereof.

### EXAMPLES

Unless otherwise stated, all temperatures are in degrees Celsius. Also, in these examples and elsewhere, the following abbreviations have the following meanings:

| | |
|---|---|
| μm = | micron |
| avg. = | average |
| cc = | cubic centimeter (equal to 1 milliliter) |
| cP = | centipoise |
| cSt = | centistoke |
| D = | shear rate (1/S) |
| DMSO = | dimethylsulfoxide |
| EH5 silica = | fumed silica having a surface area of |
| | approximately 380 m²/g (BET) (available |
| | from Cabot Corp., Tuscola, IL., USA) |
| Eta (Greek letter, η) = | apparent viscosity (Pa * s) |
| EVOH = | ethylene vinyl alcohol copolymer |
| Formula K = | 19% EVOH (wt/vol DMSO) or 8.21% |
| | EVOH (wt/final wt); |
| | 38% (wt/final wt) Ta; and |
| | 6.175% EH5 silica (wt/final wt) |
| ft = | feet |
| g = | gram |
| kg = | kilogram |
| lb. = | pound |
| m = | meter |
| M5 silica = | fumed silica having a surface area of |
| | approximately 200 m²/g (BET) (available |
| | from Cabot Corp., Tuscola, IL., USA) |
| min = | minute |
| ml = | milliliter |
| mm = | millimeter |
| PA = | Parent artery |
| PAO = | Parent artery occlusion |
| ppm = | parts per million |
| psi = | pound per square inch |
| RPM = | revolutions per minute |
| s = | second |
| Ta = | tantalum |
| Tau (Greek letter, τ) = | shear stress (Pa) |
| TS-610 silica = | fumed silica having a surface area of |
| | approximately 125 m²/g (BET) (available |
| | from Cabot Corp., Tuscola, IL., USA) |
| TS-720 silica = | fumed silica having a surface area of |
| | approximately 115 m²/g (BET) (available |
| | from Cabot Corp., Tuscola, IL., USA) |
| wt/final wt = | weight per final weight of composition |
| wt/vol DMSO = | weight per volume of DMSO |
| μg/g = | parts per million |

Equipment

Unless otherwise indicated, the following equipment was employed in the examples below.

The mixer employed in the experiments was a Morehouse-Cowles (Fullerton, Calif.) Model CM-100 Lab Disperser with optical encoder and feedback system to maintain constant RPM under variable load. The mixer had a 1.25 inch diameter hi-vane impeller Cowles blade in a 250 ml or 500 ml beaker. There was approximately a 2 to 1 or 3 to 1 ratio of beaker diameter to blade diameter respectively. The blade height, measured from about the bottom of the beaker is one half to one full blade diameter.

In order to obtain viscosity measurements, a Brookfield Engineering (Middleboro, Mass.) R/S-CPS Rheometer with RS232 interface to Windows-based PC running Brookfield Rheocalc v2.7 software. This is a cone and plate system, with two cones employed. One cone was 50 mm with 1° angle and the other cone was 75 mm with a 1° angle. There was also a Brookfield TC-501 water bath employed for temperature control to the R/S.

Compositions

In the following examples and procedures, the DMSO is USP grade. The tantalum is Q2 Grade NRC Capacitor grade tantalum metal powder from H. C. Starck (Newton, Mass.).

### Example 1

### Preparation of Compositions

This example evaluated the density, precipitation and cytotoxicity of non-sterilized polymeric embolic compositions comprising silica as a rheological modifier as compared to non-sterilized embolic composition not containing fumed silica.

Two different rheologically-modified polymeric embolic compositions were evaluated relative to two conventional polymeric embolic compositions. Specifically, two conventional embolic compositions were prepared (based on 100 ml of DMSO) as follows:

Composition A:

1) 20 g EVOH (48 percent ethylene-average molecular weight of approximately 100,000);

2) 82 g tantalum; and

3) 100 mL DMSO.

Composition B:

1) 30 g EVOH (48 percent ethylene-average molecular weight of approximately 100,000);

2) 123 g tantalum; and

3) 100 mL DMSO.

The DMSO and the EVOH were combined and the resulting composition was covered and heated to about 55±5° C. for 1.0 hours while stirring the composition. The heating was continued at the indicated temperature until all of the EVOH was dissolved. In a stirred vessel, tantalum powder was added to the EVOH and DMSO composition over a period of about 30 minutes and stirring was continued for another 20 minutes to ensure homogeneity.

Two rheologically-modified polymeric embolic compositions were prepared in a manner similar to that described above with the exception that each of the components was treated in the manner set forth in Scheme 1 prior to addition and further, after addition of the tantalum, silica was added to the composition under ambient conditions and an argon atmosphere. Each composition was initially stirred at low RPM (less than about 1000 RPM) to wet the surface of the silica. Once wetted, the stir rate was increased to a peripheral tip speed of about 20 m/sec. This tip speed was maintained until no granular material was evidenced in composition. This procedure was maintained for 5 minutes and any granular material adhering to the vessel walls was scraped into the fluid composition. Mixing as per above was then continued for another 5 minutes. This tip speed was maintained until no granular material was evidenced in composition.

The blade was positioned between 0.5 and 1 blade diameter above the bottom of the mixing vessel. The diameter of the mixing vessel was about 2 to 3 times the diameter of the mixing blade.

Upon completion of mixing of the silica-containing embolic compositions, the compositions comprised the following (based on 100 mL of DMSO)

Composition C:

1) 11 g EVOH (48 percent ethylene-average molecular weight of approximately 100,000);

2) about 82 g tantalum;

3) 100 mL DMSO; and

4) about 14 g M5 silica.

Composition D:

1) 11 g EVOH (48 percent ethylene-average molecular weight of approximately 100,000);

2) about 82 g tantalum;

3) 100 mL DMSO; and

4) about 14 g EH5 silica.

Densities were determined by conventional means.

A portion of each of the compositions was precipitated in saline. The resulting precipitate was washed twice with saline and then employed in the art recognized ISO-10993 cytotoxicity testing.

TABLE 1
lists the results of this evaluation.

| | Density | Cytotoxicity |
|---|---|---|
| Sample | (g/ml) | Result |
| Composition C | 1.764 | pass |
| Composition D | 1.778 | pass |
| Composition A | 1.73 ± 0.10 | pass |
| Composition B | 1.971 | pass |

### Example 2

### Mixing Parameters of Fumed Silica Embolic Compositions

Mixing parameters were studied.

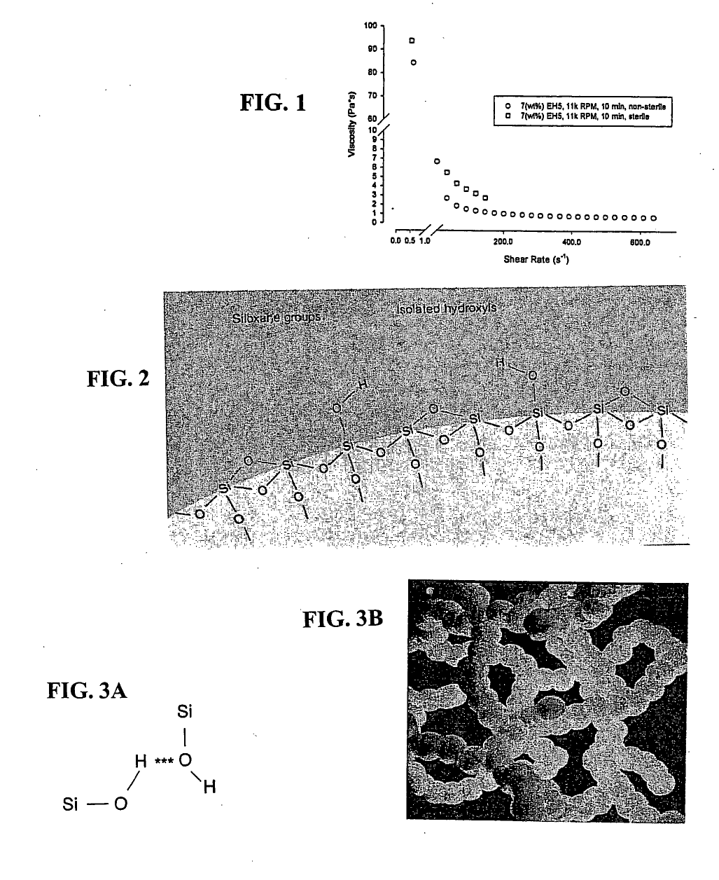

FIG. 1 illustrates non-sterile and sterile viscosities of the composition D of Example 1 versus shear rate. The composition was 10% EVOH (wt/vol DMSO), 38% Ta (wt/final wt), and 7% EH5 silica (wt/final wt) and was mixed with a 1.25″ Cowles Blade in a 500 ml beaker at the indicated RPM. FIG. 1 shows that 11,000 RPMs for 10 min produced a non-sterile fluid that was high viscosity at low shear rate and low viscosity at high shear rate (i.e. shear thinning). Upon sterilization, the material shows an increase in high and low shear viscosity

Quantification of Rheologically-modified Composition Properties

Using the Brookfield R/S-CPS Rheometer, non-sterile formulations were analyzed as in FIG. 1, measuring viscosity and shear stress against shear rate.

The shear stress data were plotted using the method of Casson (Casson (1959) Rheology of Dispersed Systems, Pergamon, N.Y.). The Casson equation (see equation 1) gives a straight line with a y-intercept that is the square root of the fluid's yield stress (K₀) and a slope (K₁) that represents the fluid viscosity at infinite shear rate.

\sqrt{τ}=K₀+K₁\sqrt{γ} Equation 1

The viscosity data were plotted using a Power Law method (Braun & Rosen (2000) Rheology Modifiers Handbook: practical use and application. William Andrew Publishing, NY). This method often gives data that can be separated into linear segments that are analyzed separately (see Equation 2).

1nη=1nK₁+K₂1nγ Equation 2

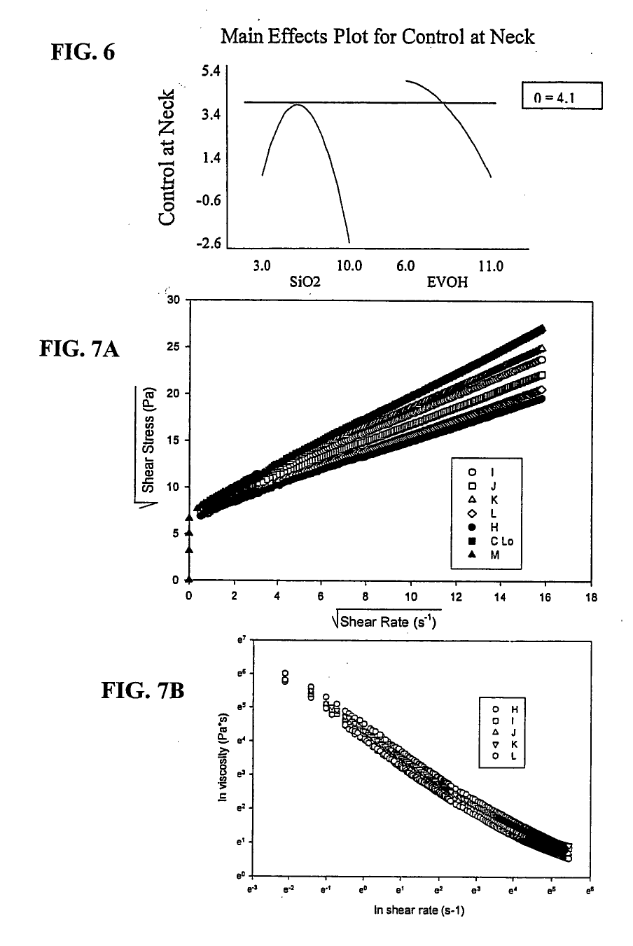

FIGS. 7A and 7B show various viscosity property data for various compositions of this invention analyzed by the Casson and Power Law methods, respectively. FIG. 7A shows the formulations in a Casson plot. FIG. 7B shows the formulations in a Power Law method.

Based on the above analysis, four rheologically-modified compositions employing the following ratios of amorphous fumed silica (silica) as the rheological modifier and ethylene vinyl alcohol copolymer (EVOH) as the biocompatible polymer were determined to have the best balance of yield stress (Casson plot in FIG. 7A) and viscosity at rest and shear thinning (Power Law plot in FIG. 7B), with K being the best:

Formula I: 6.25% silica (wt/final wt) and 7.83% EVOH (wt/final wt)

Formula J: 6.28% silica (wt/final wt) and 7.41% EVOH (wt/final wt)

Formula K: 6.175% silica (wt/final wt) and 8.21% EVOH (wt/final wt)

Formula L: 6.38% silica (wt/final wt) and 6.98% EVOH (wt/final wt)

### Example 3

### Formulation Optimization

The purpose of this testing was to evaluate and rank the in vitro, aneurysm embolization performance of several prior art formulations and formulations of rheologically-modified composition in order to optimize the formulation. All embolizations were performed in silicone lateral wall aneurysm models. The primary focus was on determining the ability of the formulations to effectively and consistently fill and model the neck of the aneurysms in a controllable fashion.

Multiple formulations were created with variable parameters of their main constituents (e.g., % EVOH (wt/vol DMSO) and % silica (wt/final wt)). These formulations were evaluated numerically on observed effects during embolization.

The numerical results were then compiled and analyzed. The analysis was used to optimize the combination of input variables (% silica/% EVOH) versus “Degree of control at the neck” primarily.

Results and Discussion

FIG. 6 illustrates a main effects plot showing contributing curves of the input variables (wt/final wt) for control at the neck. The horizontal line represents the highest average performance generated for the neck control response variable. As can be seen, there was a optimal addition of silica, in terms of control of the leakage of composition out of the neck. Similarly, control could be optimized for the amount of polymer. This proves that the control of silica addition and the proper selection of silica and polymer levels can produce a superior embolizing composition. Confirmatory tests produced results which confirmed that Formula K, which followed this optimization, gave virtually no leakage at the neck prior to aneurysm fill.

As noted, Formula K provides the optimal combination of precipitation and flow characteristics yielding the desired control at the neck

In vitro Aneurysm—Failure Modes

Silicone aneurysms were embolized with Composition A, as described in Example 1, and Formula K to compare quantitatively the volume of material that produces a parent artery protrusion. The volume that completely fills the aneurysm was measured and then the volume that creates a parent artery protrusion was measured. The ratio of volume that fills the aneurysm versus the volume that create parent artery protrusion is a measure of the material's ability to fill the aneurysm but not leak from the aneurysm. After testing Formula K against a known composition, it was confirmed that Formula K illustrates the optimal combination of precipitation and flow characteristics yielding the desired control at the neck.

The material was also tested for stability after aging. The results indicated that the there was no change in the composition's viscosity properties over time.

### Example 4

### In Vivo Confirmation

Methods

In general, all in vivo embolizations were performed per the procedure described in the canine model below. The performance was measured using the same evaluation system described in the previous example.

A 10-15 kg mongrel dog was anesthetized. Under sterile conditions and with the aid of an operating microscope, an experimental aneurysm was surgically created in the carotid artery using a jugular vein pouch, employing the method of German et al. After about one week, the aneurysm was embolized with a rheologically-modified composition.

Specifically, the femoral arteries are accessed by cut down and introducers and 7 Fr guiding catheters were placed.

For deposition of the rheologically-modified composition, a microcatheter (e.g., Micro Therapeutics, Inc. Titan, with guide wire) was placed through the guiding catheter and was positioned under fluoroscopic guidance so that the catheter tip was in the aneurysmal sac. A microballoon catheter (4-5 mm balloon) was placed in the carotid artery proximal to the aneurysm. Position was confirmed with injection of liquid contrast agent. The balloon was inflated to slow or arrest blood flow to prevent displacement of the rheologically-modified composition comprising fumed silica during injection.

Approximately 0.3 to 1 cc of sterilized rheologically-modified composition, as described in Example 3, was injected into the aneurysm over about 30 minutes to fill the aneurysm space. Care was given not to overfill the aneurysm and block the parent artery with polymer. Filling was easily visualized with fluoroscopy due to the presence of contrast agent in the polymer composition. After about 10 minutes, the polymer was fully precipitated and the catheters was removed from the artery.

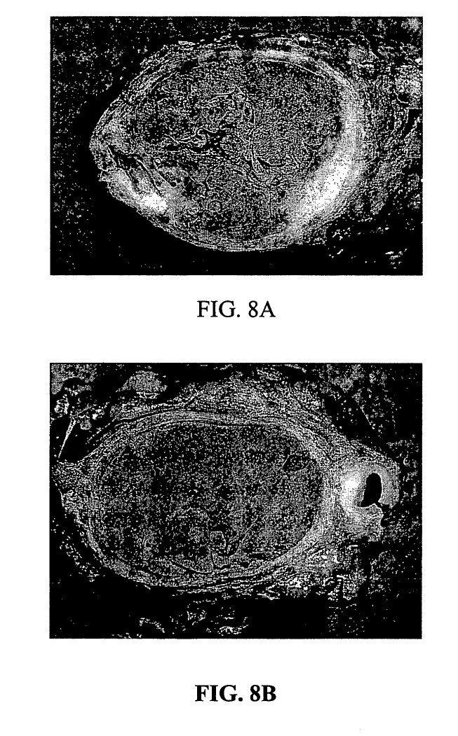

Sagittal gross pathology sections of the embolization using compositions of this invention were compared to subjects that were embolized using Composition A, not of the invention, as described in Example 1, illustrate the smooth and complete fill of the rheologically-modified composition. Results of the comparison are presented in FIGS. 8A and 8B.

The above tests provided a very positive in vivo confirmation of Formula K with no significant parent artery protrusion and high scores in the “control at the neck” response variable.

### Example 5

### Delivery System Components Development

During the development of the rheologically-modified composition, it was determined that the high shear viscosity of the Formula K is approximately 3000-4000 cP. Because of the material's high viscosity, it became desirable to modify some of the delivery system components and to incorporate others in order to handle the increased pressure demands of the material during injection. Out of this discovery, new components, evolved as follows:

These improved components, which may be used individually in combination with conventional syringe and catheter components, or together as will be described, include a vented syringe barrel, an improved connector for coupling a syringe barrel to a tube of material to be injected, a coupling for joining a syringe to a catheter and an improved mechanism, called a “Quick Stop” for easily and repeatedly engaging a lead screw mechanism to extrude the viscous composition out of the syringe and disengaging the lead screw to halt the flow of composition.

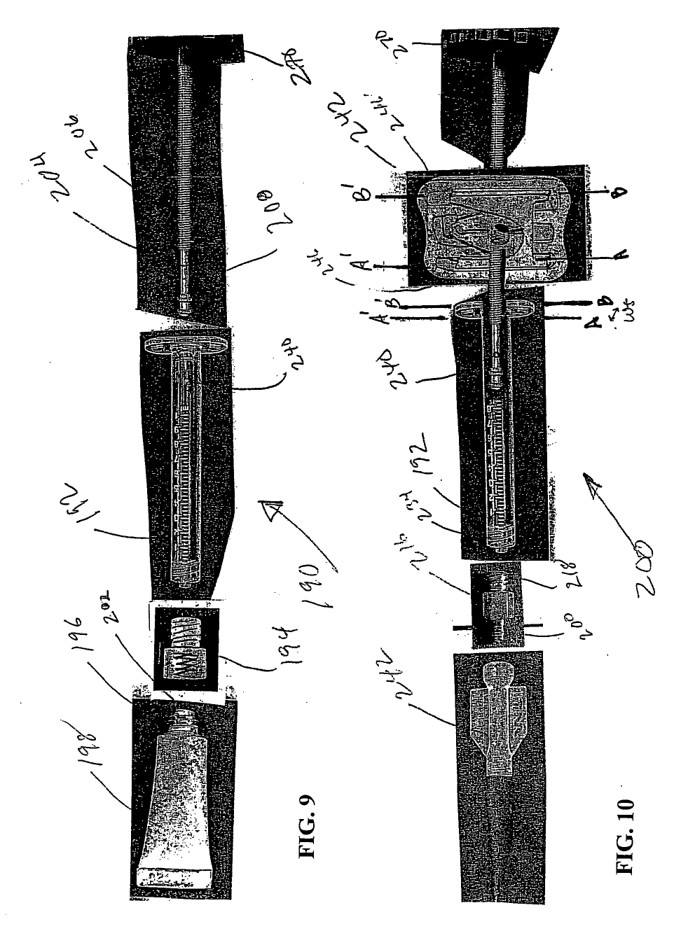

Turning to FIG. 9, a combination of components making up a system 190 suitable for loading composition into a syringe barrel is shown in an exploded view. System 190 includes a syringe barrel 192. As illustrated in more detail in FIGS. 13 and 14, syringe barrel 192 includes a distal delivery orifice 232 equipped with a male luer fitting 234. The proximal end includes an orifice 236 and a flange or handle 238. Flange 238 is typically a flat-sided oval having a width “Wf”. This width will come into play when the syringe is used in conjunction with the Quick Stop mechanism as will be described hereinafter.

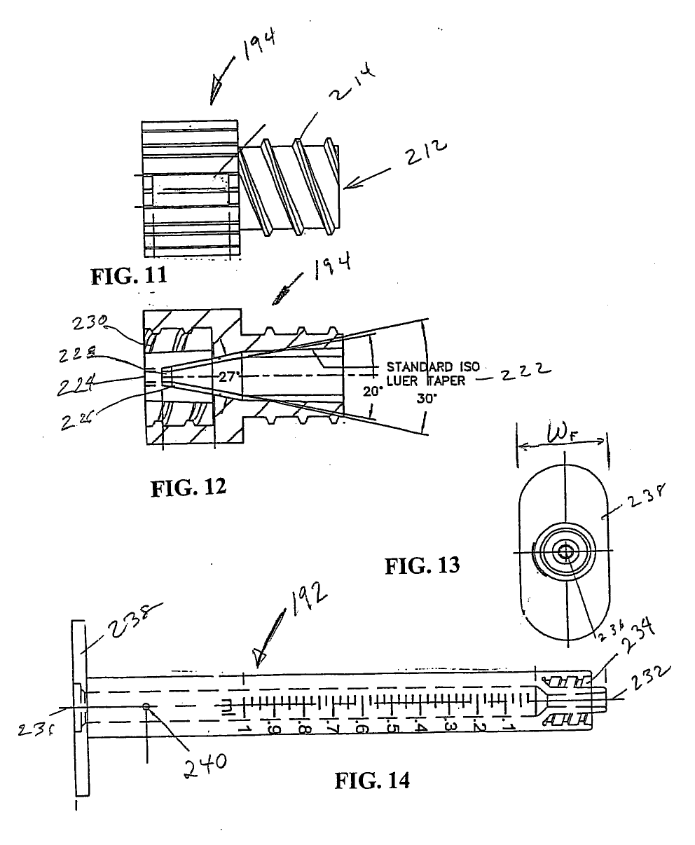

Combination 190 includes a syringe interface 194. As shown in FIGS. 11 and 12 this interface includes a proximal orifice 212 which includes threads 214 and standard luer taper 222 of a female luer fitting to sealably engage the male luer fitting on syringe barrel 192. Interface 194 also included as distal orifice 224. Orifice 224 includes internal threads 230 which are sized to correspond to match the external threads 196 present on tube 198. Tube 198 is constructed of a flexible material such as plastic or aluminum and has a orifice 202 which is typically covered with a frangible seal (not specifically shown) stretched over it. Interface 194 includes a hollow tapered barb 226 with internal opening 228 in fluid communication with orifice 212. In use, interface 194 is typically screwed into syringe body 192 and screwed onto tube 198. As interface 194 is tightened onto tube 198, via threads 196, this causes tapered barb 226 to contact and pierce the seal on tube 198 and establish a fluid flow path out of the tube, through the interface and into the syringe barrel. By squeezing tube 198, its contents, for example a relatively viscous embolic composition, can be loaded into the syringe barrel 192.

Syringe barrel 192 is equipped with at least one vent hole 240. Vent hole 240 is provided to allow air to be removed from the syringe barrel and assure a bubble-free fill of the barrel with the viscous embolizing composition or a like viscous injectable. In use, the injectable material is filled into the syringe barrel at least to the vent hole. Then when a plunger, either a conventional plunger or a plunger such as plunger 204 having threads 206 on its shaft 208 is inserted in the proximal end of the syringe body, any air that is trapped between the body of injectable composition and the syringe plunger is exhausted out through the vent hole 240. It will be appreciated that with conventional injectables which are commonly very low viscosity solutions suspensions, there is little need for vent holes. Bubbles which are trapped in a less viscous injectable can be dislodged by tapping on the syringe body and organized at the distal end of the syringe and readily expelled thought the distal orifice. This is very difficult to accomplish, if not virtually impossible, with viscous injectables such as the embolizing compositions of this invention. Accordingly, the vent holes provide a significant advantage.

Turning to FIG. 10 and referring to FIG. 9, as well, an improved system 200 for delivering a viscous injectable, and in particular the viscous embolizing compositions of this invention is shown. The viscous injectable is not shown but would have been loaded into syringe barrel 192 to a volume at least up to the vent holes 240. In use, interface 194 would be removed and replaced by adapter 216. Adapter 216 includes a female luer connector which matches and engages connector 234 on barrel 194. At its other end adaptor 216 is threaded with a thread 220 corresponding to a thread on catheter 242. Adapter 216 sealably joins catheter 242 to syringe barrel 192 and creates a fluid path between them.

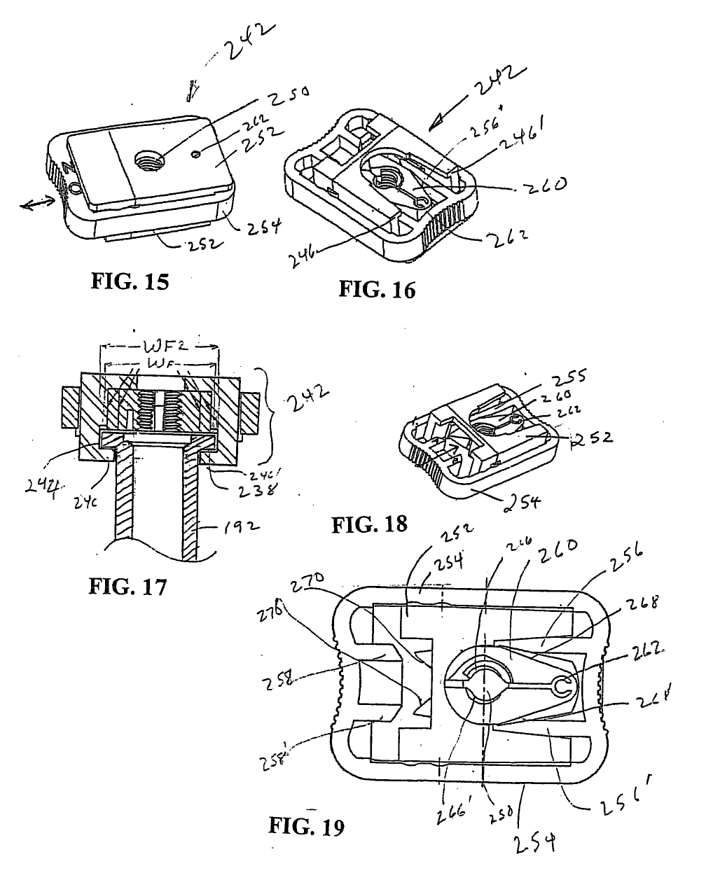

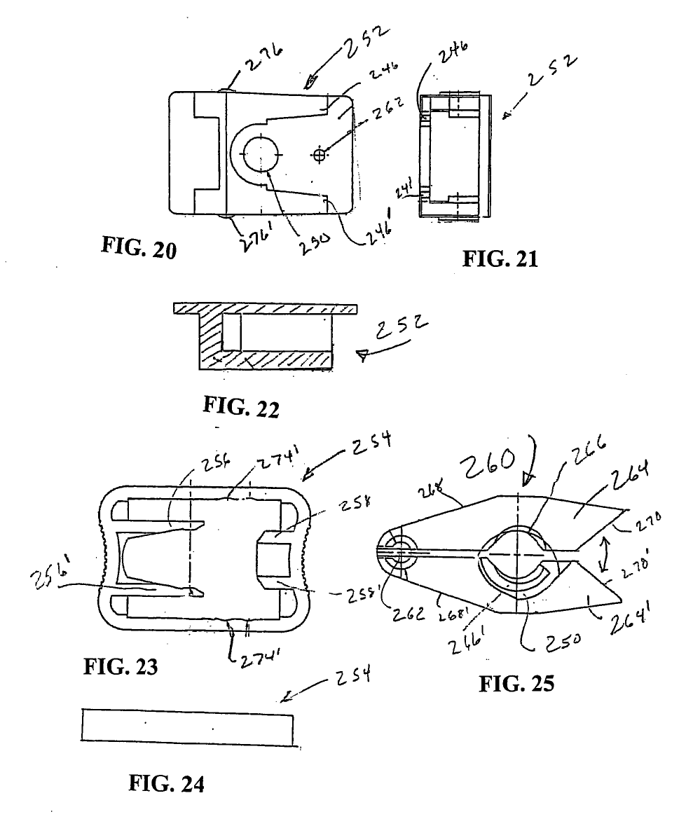

System 200 also includes Quick Stop 242. Quick Stop 242 is illustrated in detail in FIGS. 15-19 and its components are shown in FIGS. 20-25. As shown most clearly in FIG. 17, Quick Stop 242 is sized to slide over and engage the flange 238 on syringe barrel 192. As noted previously, syringe body 192 included a flange 238 which has a width Wf. Quick Stop 242 has a slot 244 defined by shoulders 246 and 246′ which is somewhat wider than flange 238. Thus, as shown in FIG. 10, when Quick Stop 242 is slid along flange 238 with its lines A-A′ and B-B′ corresponding to the same lines on the flange 238, the two parts engage as shown in FIG. 17. As can be seen in FIG. 10, and in more detail in FIGS. 16 and 18, shoulder 246 and 246′ not only define a slot in which the syringe barrel 192 is inserted, they also extent inward toward each other and join at region 24. This region limits the insertion of the flange 238 of syringe barrel 194 to a predetermined distance. This predetermined distance is such that threaded hole 250 is axially aligned with barrel 192.

As shown in FIG. 15's perspective top view, hole 250 is contained in base 252. Activator 254 slides back and forth, relative to base 252, along the same direction that barrel 193 slid into slot 244. FIG. 24 depicts actuator 254 and shows that it has two pairs of inwardly-extending fingers, 256 and 256′ and 258 and 258′. These fingers bear upon surfaces of a part known as threaded pincher 260. FIG. 25 shows that 260 has a pivot point 262 and has two wings 264 and 264′ which can move together and apart relative to one another. Wings 264 and 264′ each contain a portion of a threaded aperture 266 and 266′. When the two wings are brought together they define a nearly complete threaded cylinder which is matched to the thread 208 present on threaded syringe plunger shaft 206. By “nearly complete” it is meant that at least about 280° of the full circle are created and preferably at least 300° and especially at least about 320° degrees. This extent of circle creation is important as it assures that there is a solid grip in the threaded shaft 208 of plunger 204 when the threaded pincher is closed around it and it permits greater forces to be exerted on the plunger 204 to extrude the high viscosity embolizing compositions.

As can be seen most clearly in FIGS. 18 and 19, actuator 254 is moved as far to the left as possible relative to base 252. This has caused fingers 256 and 256′ to bear against surfaces 268 and 268′ on threaded pincher 260 and this in turn has caused threads 266 and 266′ to form a threaded aperture in its “on” or “engagement” mode. If threaded plunger shaft 208 was in place in aperture 250, this would result in threads 266 and 266′ engaging the threads 206 on shaft 208. When so engaged, threads 206 on shaft 208 would function as a lead screw when rotated such as by knob 270. This would cause plunger 204 to move inward or outward relative to syringe body 192, depending upon the direction of rotation of knob 270. This permits the gradual and controllable delivery of the viscous composition out of the syringe barrel to the location of use such as through catheter 242. If activator 254 were slid to the right, this would release the pressure or fingers 256 and 256′ on surfaces 268 and 268′, respectively and would, instead cause fingers 258 and 258′ to engage and push apart surfaces 270 and 270′ on threaded pincher 360. This would cause the threaded aperture to “open”. This would create an opening though which plunger 204 could be inserted. It would also release the grip in threaded plunger shaft 208, if the plunger were in place passing through hole 250 into barrel 192. When this grip is released it would instantaneously release the pressure e on the viscous material in the syringe barrel and halt the material's delivery such as through catheter 242.

As seen most clearly in FIGS. 20 and 23, base 252 and activator 254 may be equipped with one or more detent mechanisms to allow the device to be temporarily locked in the “on” or “engaged” position. This can be accomplished with, for example indents 274 and 274′ which engage extensions 276 and 276′. This locking mechanism can prevent inadvertent releases of pressure as might occur if the actuator were permitted to freely move out of the on position.

### Example 5A

### Quick Stop Clip for the Threaded Injector

This is the new cam-slide mechanism that incorporates an approximate 340° thread engagement as opposed to the known Quick Stop as described in United States Patent Application Publication No. 2003-0055386, which only engaged approximately 220° of the plunger thread. In addition, this new mechanism reduces, significantly, the amount of force necessary to engage and disengage the plunger.

The static pressure capability of the new clip was tested and the average peak pressure (psi) was 2,731.

The force to engage and disengage threads was also tested compared with the old clip. When completing 10 cycles, the average force (lb.) was to engage the threads was 3.45, as compared to 8.48 for the old clip. To disengage the threads, an average force of 3.28 was required, compared to 8.18 for the old clip.

Based on the above results, the new Quick Stop is a significant improvement over the prior mechanism. The force required to engage and disengage the new clip is less than half the original. In addition, the pressure capability of the new clip has been increased by approximately 1000 psi.

Schematic drawings of the Quick Stop mechanism are shown in FIG. 10 and FIG. 15 through FIG. 25.

### Example 5B

### Stainless Steel Syringe-to-catheter Interface

The peak pressure of the interface at a given flow rate of 0.1 ml/min, 0.2 ml/min., 0.3 ml/min, and 0.5 ml/min was tested. The average pressures (psi) were as follows: at 0.1 ml/min, 948 psi; at 0.2 ml/min, 1520 psi; 0.3 ml/min., 1829; and 0.5 ml/min, 1897.

Based on the above results, the new interface fitting 216 is a significant improvement over the current HD injector connection. The pressure capability of the new fitting has been increased by approximately 400 psi.

### Example 5C

### Modified Threaded Plunger

The modified plunger (204 in the drawings) employs a change to the diameter of the piston head before and after the O-rings for the purpose of increasing its pressure capability. The prior design can withstand approximately 1700-1900 psi before the O-rings would begin to peel back and fail. By increasing the surface area (diameter) of the leading head on the piston and by increasing the diameter of the piston body behind each o-ring, the pressure capability of the plunger could be significantly increased. When testing the static pressure capacity of the new Quick Stop clip, the modified plunger was used.

### Example 5D

### Puncture Cap with Standard Luer Interface

With the new aluminum tube packaging described herein comes the need to be able to puncture the tube membrane and transfer the material to the delivery syringe. To meet this need, a suitable puncture cap with luer interface 194 was designed. This device is a small cap. As described above, one end screws onto the tube while creating a burrless puncture hole in the membrane and the other end has a ISO luer fitting that can be fitted to the tip of the threaded injector for the composition transfer.

Functionality Test

The new cap effectively and cleanly punctured the tube membrane and connected to the delivery syringe with ease as designed.

### Example 6

### Sterilization and Stability

Sterilization

During the initial evaluation of the composition, a noticeable change in the fluid occurred during heat sterilization. The fluid viscosity increased and the shear stress vs. shear rate analysis showed a time-dependent behavior known as thixotropy.

As noted previously, non-sterile rheologically-modified composition shows little or no deviation in shear stress as the shear rate is varied from 0 to 250 s⁻¹ and from 250 to 0 s⁻¹. From the same rheologically-modified composition mix, a sample of heat sterilized composition shows much higher shear stress over 0 to 250 s⁻¹ then from 250 to 0 s⁻¹. This deviation, or hysteresis in shear stress as shear rate is increased then decreased, is thixotropy. The composition used had the following formulation: 18% EVOH (wt/vol DMSO); 38% Ta (wt/final wt); 6.25% silica EH5 (wt/final wt) and the balance DMSO.

Several tests were conducted to understand what chemical entities changed to promote the thixotropy. Thixotropy is directly related to surface silanol concentration. A hypothesis was that silanol groups from different silica particles and/or the biocompatible polymer become covalently bonded together rather than hydrogen bonded and this new structure is more viscous and requires more shear stress to flow. Cabot Corporation sells partially treated and completely treated fumed silica. The partial treatment removes most but not all of the silanol groups from the silica particle surface. The completely treated silica contains no reported surface silanols.

In order to evaluate the above, the reduction in thixotropy was determined by using partially treated silica in the rheologically-modified composition and the elimination of thixotropy using completely treated silica. Specifically, shear stress vs. shear rate for a RM composition, non-sterile and sterile, with untreated, partially treated, and completely treated silica. The following data was obtained:

| Rheological Modifier/ | Thixotropy | Increase from |
|---|---|---|
| Condition | (Pa/s) | non-sterile (fold) |
| Untreated silica/Non-sterile | 4,657 | Not Applicable |
| Untreated silica/Heat sterile | 31,058 | 6.669 |
| Partially treated silica/Heat sterile | 10,948 | 2.351 |
| Completely treated silica/Heat sterile | 3,485 | 0.7483 |

The shear stress curves show a partial reduction in thixotropy by the partially treated silica and complete reduction in thixotropy by completely treated silica. rheologically-modified composition formulations were: Untreated and partially treated=19% EVOH (wt/vol DMSO), 38% Ta (wt/final wt), 6.125% EH5 or TS-610 silica (wt/final wt); Completely treated=19% EVOH (wt/vol DMSO), 38% Ta (wt/final wt), 3% TS-720 silica (wt/final wt).

Stability

Given the change in material due to sterilization, preliminary shelf life and thermal cycling data were collected and the results indicated that the viscosity properties of the compositions were not significantly altered. The physical properties and embolization performance characteristics of Formula K remain stable and consistent after 6 months accelerated aging within the aluminum tube packaging.

### Example 7

### E-Beam Sterilization of Formula K in an Aluminum Tube

Formula K, a described above, was injected into 3 mL aluminum tubes (30 in all) also as described above. The 30 tubes were divided into three groups of 10, 8 subject to e-beam sterilization and two serving as controls. The first group, other than the control samples, was subjected to e-beam sterilization at a total dosing of 20 kGy. The second group, other than the control samples, was subjected to e-beam sterilization at a total dosing of 40 kGy. The third group, other than the control samples, was subjected to e-beam sterilization at a total dosing of 60 kGy.

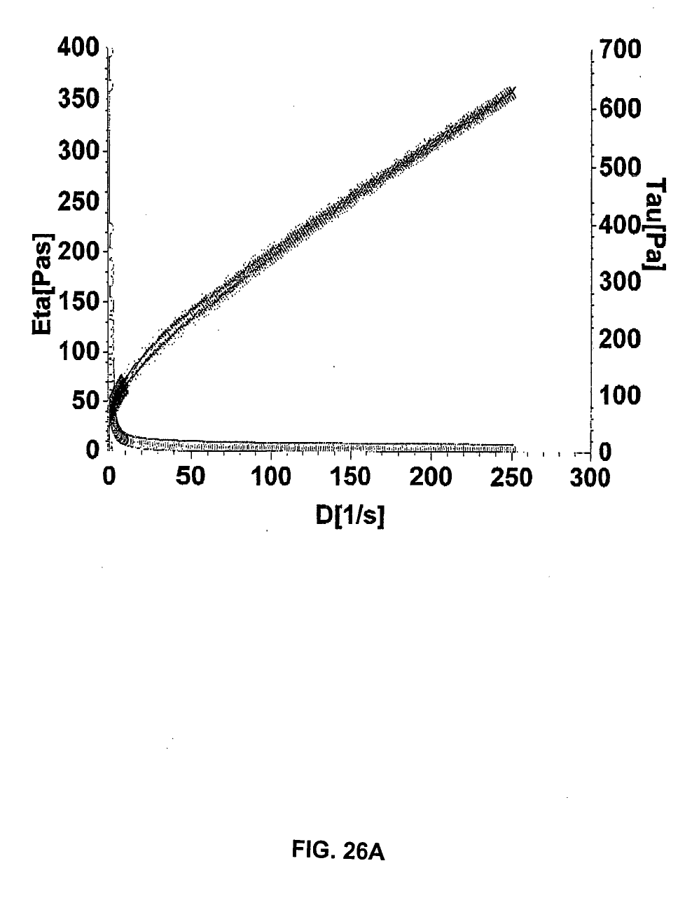

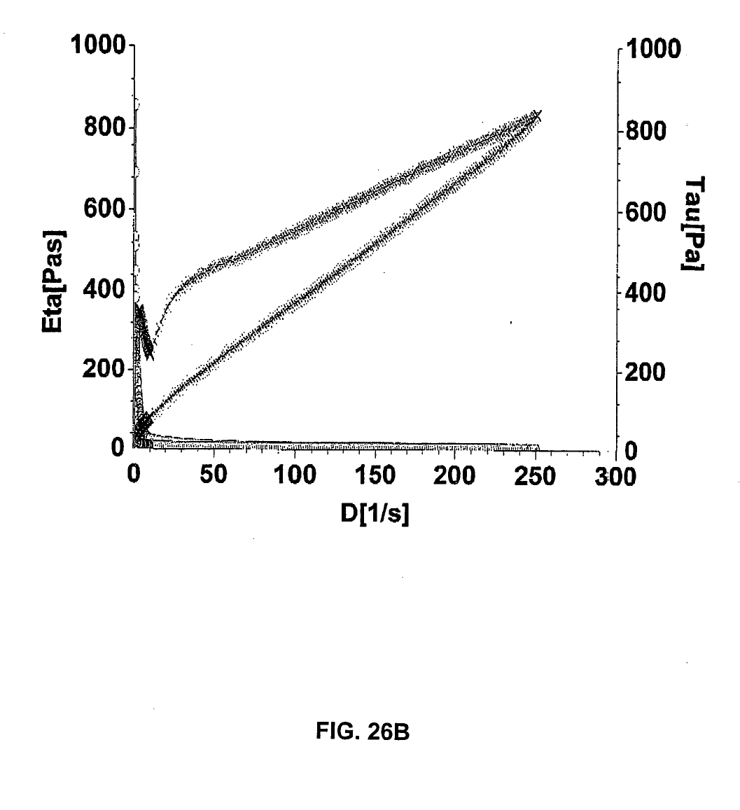

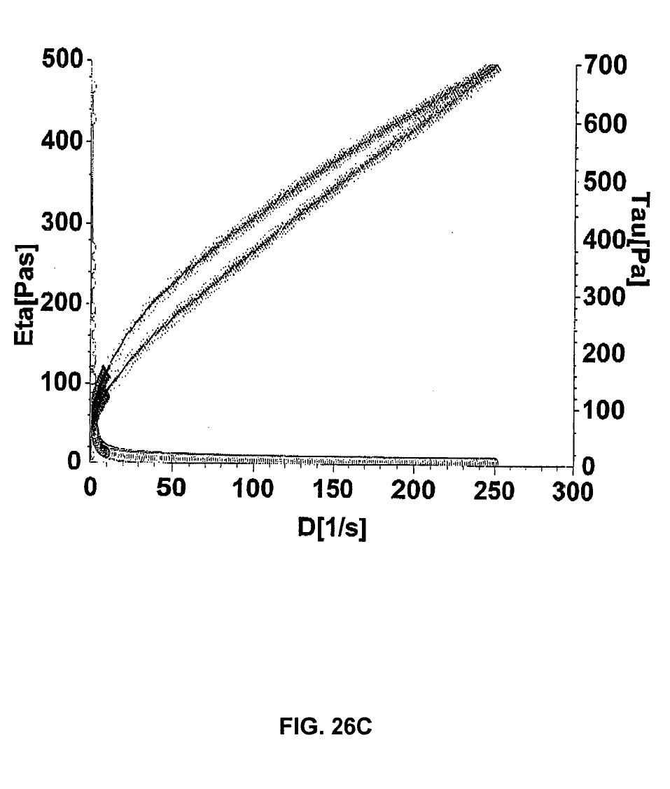

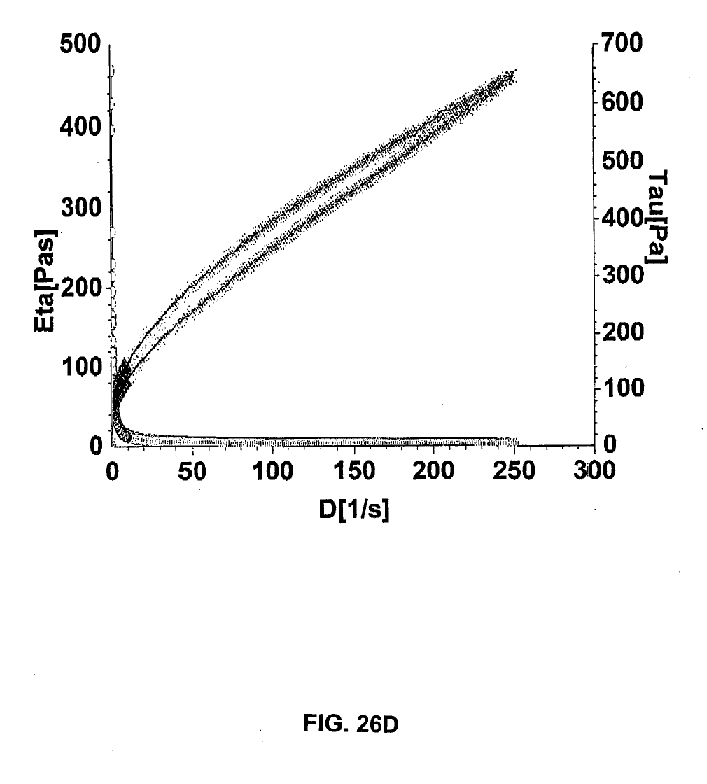

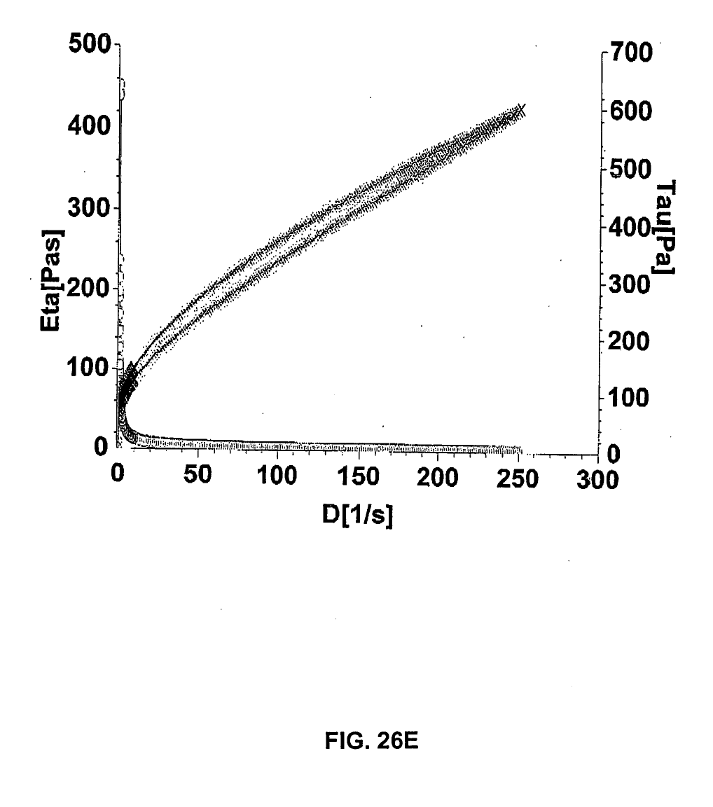

FIGS. 26A-E demonstrate rheology profile for the each of the compositions compared to control and heat sterilization. As is apparent, there is little change in the rheological profile of the e-beam sterilized compositions relative to control. Contrarily, the heat sterilized composition exhibits significant deviation in its rheological properties as compared to control.

The table below illustrates the area between the two curves measuring shear stress at increasing and decreasing shear rates measured at from 0 to 250 s⁻¹ for each of these compositions:

| | |
|---|---|
| Control | 2,248 Pa/sec |
| E-beam sterilized | 10,684 Pa/sec |
| Heat Sterilized | 36,880 Pa/sec |

The above data demonstrates that the e-beam sterilized compositions of this invention retain pseudo-plastic properties similar to control whereas the heat sterilized compositions did not.

From the foregoing description, various modifications and changes in the composition and method will occur to those skilled in the art. All such modifications coming within the scope of the appended claims are intended to be included therein.

What is claimed is:

1. A method for sterilizing an embolic composition comprising a hydroxyl-containing rheological modifier in an effective amount to impart shear thinning, pseudo-plastic properties to the composition which method comprises exposing the composition to a sufficient amount of irradiation to effect sterilization under conditions such that the sterilized composition exhibits a minimal change in its thixotropic behavior as compared to the composition prior to sterilization wherein such minimal change is characterized by an area between the two curves measuring shear stress at increasing and decreasing shear rates measured at from 0 to 250 s⁻¹ of no more than about 25,000 Pa/sec.

2. The method according to claim 1, wherein the area between the two curves is from about area 1,000 to about 20,000 Pa/sec.

3. The method according to claim 2, wherein the area between the two curves is from about 2,500 to about 15,000 Pa/sec.

4. The method according to claim 1, wherein the irradiation is gamma irradiation.

5. The method according to claim 1, wherein the irradiation is electron beam irradiation.

6. The method according to claim 1, wherein the hydroxyl-containing rheological modifier is amorphous, fumed silica.

7. A sterilized embolic composition comprising a hydroxyl-containing rheological modifier in an effective amount to impart shear thinning, pseudo-plastic properties to the composition wherein the sterilized composition exhibits a minimal change in its thixotropic behavior as compared to the composition prior to sterilization wherein such minimal change is characterized by an area between the two curves measuring shear stress at increasing and decreasing shear rates measured at from 0 to 250 s⁻¹ of no more than about 25,000 Pa/sec.

8. The sterilized embolic composition according to claim 7 wherein the area between the two curves is from about area 1,000 to about 20,000 Pa/sec.

9. The sterilized embolic composition according to claim 8 wherein the area between the two curves is from about area 2,500 to about 15,000 Pa/sec.

10. The sterilized embolic composition according to claim 7, wherein the sterilized composition is further characterized by exhibiting an increase of less than about 25% of its viscosity at 37° C. over a shelf-life of 6 months or more at a high shear of 250 sec⁻¹ as compared to the viscosity under the same conditions immediately after sterilization.

11. The sterilized embolic composition according to claim 10, wherein the sterilized composition is further characterized by exhibiting an increase of less than about 20% of its viscosity at 37° C. over a shelf-life of 6 months or more at a high shear of 250 sec⁻¹ as compared to the viscosity under the same conditions immediately after sterilization.

12. The sterilized embolic composition according to claim 11, wherein the sterilized composition is further characterized by exhibiting an increase of less than about 15% of its viscosity at 37° C. over a shelf-life of 6 months or more at a high shear of 250 sec⁻¹ as compared to the viscosity under the same conditions immediately after sterilization.

13. The sterilized embolic composition according to claim 12, wherein the sterilized composition is further characterized by exhibiting an increase of less than about 10% of its viscosity at 37° C. over a shelf-life of 6 months or more at a high shear of 250 sec⁻¹ as compared to the viscosity under the same conditions immediately after sterilization.

14. The sterilized embolic composition according to claim 7, wherein the composition further comprises a water insoluble, biocompatible polymer, a biocompatible solvent which dissolves the biocompatible polymer in the amounts employed and optionally a visualizing effective amount of a contrast agent.

15. The sterilized embolic composition according to claim 14, wherein the composition, in the absence of the rheological modifier, has a viscosity of at least 150 cP at 37° C.

16. The sterilized embolic composition according to claim 15, wherein the composition, in the absence of a rheological modifier, has a viscosity of at least 100 cP at 37° C.

17. The sterilized embolic composition according to claim 7, wherein the composition further comprises a prepolymer and a visualizing effective amount of a contrast agent wherein the prepolymer, upon polymerization, forms a water insoluble, biocompatible polymer.

18. The sterilized embolic composition according to claim 17, wherein the composition has a viscosity of no more than 150 cP at 37° C. in the absence of a rheological modifier.

19. The sterilized embolic composition according to claim 17, wherein the composition has a viscosity of no more than 100 cP at 37° C. in the absence of a rheological modifier.

20. The sterilized embolic composition according to claim 7, wherein the hydroxyl-containing rheological modifier is amorphous, fumed silica.

21. A method for sterilizing an embolic composition comprising a hydroxyl-containing rheological modifier in an amount sufficient to impart shear thinning, pseudo-plastic properties to the composition, which method comprises exposing the composition to a sufficient amount of heat or irradiation to effect sterilization under conditions wherein the sterilized composition exhibits a minimal increase in its thixotropic behavior as compared to the composition prior to sterilization which method comprises selecting an embolic composition comprising a hydroxyl-containing rheological modifier wherein at least about 25% of the surface hydroxyl groups have been converted to non-hydroxyl groups and sterilizing said composition such that the sterilized composition exhibits a minimal change its thixotropic behavior as compared to the composition prior to sterilization which such minimal change is characterized by an area between the two curves measuring shear stress at increasing and decreasing shear rates measured at from 0 to 250 s⁻¹ of no more than about 25,000 Pa/sec.

22. The method according to claim 21 wherein the area between the two curves is from about 1,000 Pa/sec to about 20,000 Pa/sec.

23. The method according to claim 22 wherein the area between the two curves is from about 2500 Pa/sec to about 15,000 Pa/sec.

24. The method according to claim 21, wherein at least about 50% of the surface hydroxyl groups have been converted to non-hydroxyl groups.

25. The method according to claim 24, wherein at least about 90% of the surface hydroxyl groups have been converted to non-hydroxyl groups.

26. The method according to claim 25, wherein at least about 98% of the surface hydroxyl groups have been converted to non-hydroxyl groups.

27. The method according to claim 21, wherein the sterilized composition is further characterized by exhibiting a reduction of less than about 25% of its viscosity over a 1 year shelf-life.

28. The method according to claim 27, wherein the sterilized composition is further characterized by exhibiting a reduction of less than about 15% of its viscosity over a 1 year shelf-life

29. The method according to claim 21, the hydroxyl-containing rheological modifier is amorphous fumed silica.

30. The method according to claim 29, wherein the sterilized embolic composition further comprises a water insoluble, biocompatible polymer, a biocompatible solvent which dissolves the biocompatible polymer in the amounts employed and optionally a visualizing effective amount of a contrast agent.

31. The method according to claim 30, wherein the embolic composition, in the absence of the rheological modifier, has a viscosity of at least 150 cP at 37° C.

32. The method according to claim 31, wherein the embolic composition, in the absence of a rheological modifier, has a viscosity of at least 10,000 cP at 37° C.

33. The method according to claim 21, wherein the sterilized embolic composition further comprises a prepolymer and a visualizing effective amount of a contrast agent.

34. The method according to claim 33, wherein the embolic composition, in the absence of the rheological modifier, has a viscosity of no more than 150 cP at 37° C.

35. The method according to claim 34, wherein the embolic composition, in the absence of a rheological modifier, has a viscosity of no more than 100 cP at 37° C.

36. A sterilized embolic composition comprising a sufficient amount of a hydroxyl-containing rheological modifier to impart pseudo-plastic, shear thinning properties to the composition wherein at least about 25% of the surface hydroxyl groups have been converted to non-hydroxyl groups and further wherein said sterilized composition exhibits a minimal change in its thixotropic behavior as compared to the composition prior to sterilization wherein such minimal change is characterized by an area between the two curves measuring shear stress at increasing and decreasing shear rates measured at from 0 to 250 s⁻¹ of no more than about 25,000 Pa/sec.

37. The sterilized embolic composition according to claim 36 wherein the area between the two curves is from about 1,000 to about 20,000 Pa/sec.

38. The sterilized embolic composition according to claim 37 wherein the area between the two curves is from about area 2,500 to about 15,000 Pa/sec.

39. The sterilized embolic composition according to claim 36, wherein at least about 50% of the surface hydroxyl groups have been converted to non-hydroxyl groups.

40. The sterilized embolic composition according to claim 39, wherein at least about 90% of the surface hydroxyl groups have been converted to non-hydroxyl groups.

41. The sterilized embolic composition according to claim 36, wherein the sterilized composition is further characterized by exhibiting a reduction of less than about 25% of its viscosity over a 1 year shelf-life.

42. The sterilized embolic composition according to claim 36, wherein the sterilized composition is further characterized by exhibiting a reduction of less than about 20% of its viscosity over a 1 year shelf-life.

43. The sterilized embolic composition according to claim 42, wherein the sterilized composition is further characterized by exhibiting a reduction of less than about 15% of its viscosity over a 1 year shelf-life.

44. The sterilized embolic composition according to claim 43, wherein the sterilized composition is further characterized by exhibiting a reduction of less than about 10% of its viscosity over a 1 year shelf-life.

45. The sterilized embolic composition according to claim 36, wherein the sterilized embolic composition further comprises a water insoluble, biocompatible polymer, a biocompatible solvent which dissolves the biocompatible polymer in the amounts employed and optionally a visualizing effective amount of a contrast agent.

46. The sterilized embolic composition according to claim 45, wherein the embolic composition, in the absence of the rheological modifier, has a viscosity of at least 150 cP at 37° C.

47. The sterilized embolic composition according to claim 46, wherein the embolic composition, in the absence of a rheological modifier, has a viscosity of at least 100 cP at 37° C.

48. The sterilized embolic composition according to claim 36, wherein the sterilized embolic composition further comprises a prepolymer and a visualizing effective amount of a contrast agent.

49. The sterilized embolic composition according to claim 48, wherein the embolic composition, in the absence of the rheological modifier, has a viscosity of no more than 150 cP at 37° C.

50. The sterilized embolic composition according to claim 49, wherein the embolic composition, in the absence of a rheological modifier, has a viscosity of no more than 100 cP at 37° C.
